(19) United States
(12) Patent Application Publication
(10) Pub. No.: US 2014/0335102 A1
(43) Pub. Date: Nov. 13, 2014

(54) IN SILICO AFFINITY MATURATION

(71) Applicant: SANOFI, Paris (FR)

(72) Inventors: Michael Oberlin, Saint Germain Les Arpajonm (FR); Romano Kroemer, Paris (FR); Vincent Mikol, Paris (FR); Hervé Minoux, Paris (FR); Nicolas Baurin, Paris (FR)

(73) Assignee: Sanofi, Paris (FR)

(21) Appl. No.: 14/367,642

(22) PCT Filed: Dec. 18, 2012
(86) PCT No.: PCT/IB2012/002994

§ 371 Date: Jun. 20, 2014

Related U.S. Application Data
(60) Provisional application No. 61/578,527, filed on Dec. 21, 2011.

(30) Foreign Application Priority Data
Nov. 30, 2012 (FR) 1261489

(51) Int. Cl. G01N 33/53; G06F 19/12; C07K 16/40; C12N 9/22; G01N 33/573; C07K 16/28

(52) U.S. Cl. CPC G01N 33/53; G01N 33/573; C07K 16/2839; C07K 16/40; C07K 16/2863; C12N 9/22; G06F 19/12; C12Y 301/27; C07K 2317/14; C07K 2317/92; G01N 2333/922; G01N 2333/001
USPC 424/158.1; 506/9; 506/18; 536/23.53; 536/23.2; 530/389.1; 424/172.1; 435/196; 424/94.61; 435/332; 435/325; 435/334; 435/338

(57) ABSTRACT

Methods are disclosed for increasing the binding affinity of binding proteins using in silico affinity maturation.

### RELATED APPLICATIONS

This application claims priority to U.S. Provisional Application No. 61/578,527, filed Dec. 21, 2011 and French Patent Application Number 1261489 filed Nov. 30, 2012. The contents of these applications are each hereby incorporated by reference in their entireties.

### BACKGROUND OF THE INVENTION

Antibodies and other binding proteins are the subject of intense interest in pharmaceutical research. For example, monoclonal antibodies possess excellent pharmacokinetics, up to sub-nanomolar affinity, and low immunogenicity. Monoclonal antibodies have been produced by immunization of mice, construction of hybridomas, and selection of single clones expressing the desired antibody. More recently, directed evolution techniques such as phage display and related in vitro library display methods have been used. But before they can become clinical candidates, the binding affinity and other properties need to be optimized in order to maximize their chances of success during clinical development. In the field of antibody engineering, such an optimization of involves finding the right mutations to improve the property of interest. However, the preparation and characterization of all the possible mutants would imply high costs and have a significant impact on timelines, due to the combinatorial nature of the task.

### SUMMARY OF THE INVENTION

The present disclosure is based, in part, on the discovery that the affinity of a binding protein for its substrate can be improved by modifying particular amino acid residues within the binding site of the binding protein. The modifications are identified by conducting an in silico analysis of binding interactions between residues of the binding protein and the protein to which it binds. The disclosure provides methods for the selection of an appropriate modification at the identified residue position, e.g., side chain chemistry, by building a subset of modifications in silico followed by recalculating the binding free energy and election of a preferred modification. The disclosure provides a more sophisticated analysis for revealing the exact residue positions and side chain chemistries to be used to modify the binding-affinity of an antibody/antigen complex. Further, the disclosure presents methods for using computational protein engineering to guide in vitro experiments, and/or to limit the number of variants to be generated and tested subsequently in vitro or in vivo.

Previously, methods such as electrostatic optimization, side chain repacking, flexible backbone, and Dead End Elimination pruning and the A* algorithm (DEE/A*)+molecular mechanics, and a Poisson-Boltzmann surface area sampling (MM/PBSA) have been used in attempts to identify candidates for mutation through in silico affinity maturation (Clark, L. A. et al., Affinity enhancement of an in vivo matured therapeutic antibody using structure-based computational design. Protein Sci 2006, 15(5): 949-60). These previously used methods have not consistently provided useful candidates for improving the affinity binding of antibodies. Moreover, the high variability of properties when going from one system to another makes it difficult to get a general idea of the performance of a method if the dataset is not large enough. The present disclosure improves upon the prior art by using novel combinations of in silico predictors to identify mutant antibodies that have increased binding affinity for antigens. The present disclosure also uses as a novel implementation of the DEE/A*-MM/PBSA protocol and other statistical predictors to identify and test candidate antibody variants.

In one aspect, the invention provides a method of identifying a variant of an antibody with enhanced antigen binding affinity, the method comprising:

(a) determining a three-dimensional representation of an antibody-antigen interface of a first antibody,

(b) conformationally sampling point mutations of amino acid residues of the antibody at the antibody-antigen interface,

(c) selecting the point mutations of step (b) that are conformationally allowed,

(d) selecting the point mutations of step (c) that have both a Boltzmann averaged predictor based on a change of a change of a free energy of binding (ΔΔG*) of less than zero kcal/mol and a Boltzmann averaged predictor based on only a change of a change of the polar component of a free energy of binding (ΔΔEₚₒₗ*) of less than zero kcal/mol, and

(e) creating a focused library of antibodies containing at least one point mutation from the point mutations of step (d),

(f) screening the antibodies of step (e) for enhanced antigen-binding affinity in vitro; and

(g) selecting an antibody of step (f) having enhanced binding affinity relative to the first antibody, thereby identifying a variant of an antibody with enhanced antigen binding affinity.

In one embodiment, the amino acid residues of the antibody at the antibody-antigen interface that are conformationally sampled in step (b) are limited to those amino acid residues that are determined by in silico alanine screening to have an effect on the overall change in the change of free energy of binding of less than 1 kcal/mol.

In another embodiment, the amino acid residues of the antibody at the antibody-antigen interface that are conformationally sampled in step (b) are limited to those amino acid residues that are point mutations caused by at least two nucleic acid base changes in the codon coding for the point mutation of the antibody.

In another embodiment, the amino acid residues of the antibody at the antibody-antigen interface that are conformationally sampled in step (b) are limited to those amino acid residues that are point mutations that do not cause a change in the stability of the modified, mutated or altered antibody that is greater than 3 kcal/mol.

In another embodiment, the three-dimensional representation is a crystal structure having a resolution of about 2.5 Angstroms or less.

In another embodiment, wherein the point mutations of the amino acid residues of step (b) result in an alteration of amino acid side chain chemistry.

In another embodiment, the method further comprises expressing the modified, mutated or altered antibody.

In another embodiment, the method is repeated at least one time.

In another embodiment, at least one step is informed by data selected from the group consisting of binding data derived from an expressed antibody binding to an antigen in an aqueous buffer, crystal structure data of an antibody, crystal structure data of an antibody bound to an antigen, three-dimensional structural data of an antibody, NMR structural data of an antibody, and computer-modeled structural data of an antibody.

In another embodiment, the method comprises expressing the modified antibody is in an expression system selected from the group consisting of an acellular extract expression system, a phage display expression system, a prokaryotic cell expression system, and a eukaryotic cell expression system.

In another embodiment, the antibody, or antigen-binding fragment thereof, is modified at one or more CDR and/or framework positions within the light and/or heavy chain variable regions of the antibody or binding fragment.

In another embodiment, the antibody, or antigen-binding fragment thereof, is modified at one or more positions within a CDR region(s) selected from the group consisting of V_{H} CDR1, V_{H} CDR2, V_{H} CDR3, V_{L} CDR1, V_{L} CDR2, and V_{L} CDR3.

In another embodiment, the antibody, or antigen-binding fragment thereof, is selected from the group consisting of an antibody, an antibody light chain (V_{L}), an antibody heavy chain (V_{H}), a single chain antibody (scFv), a F(ab′)₂ fragment, a Fab fragment, an Fd fragment, and a single domain fragment.

In another embodiment, the antigen-binding affinity of the antibody is predicted to be increased by a factor of about 1.1, 1.2, 1.3, 1.4, 1.5, 1.6, 1.7, 1.8, 1.9, 2, 3, 5, 8, 10, 50, 10², 10³, 10⁴, 10⁵, 10⁶, 10⁷, or 10⁸.

In another aspect, the invention provides a plurality of antibodies, or antigen-binding fragments thereof, produced by the method of the invention. In another aspect the invention provides a nucleic acid encoding the antibody, or antigen-binding fragment thereof, of the invention.

In another aspect, the invention provides a host cell encoding the nucleic acid of the invention.

In another aspect, the invention provides an antibody, or binding fragment thereof, produced by culturing the host cell of the invention under conditions such that antibody, or binding fragment thereof, is expressed.

In another aspect, the invention provides a pharmaceutical composition comprising the antibody, or antigen-binding fragment thereof, of the invention.

In another aspect, the invention provides a method for treating or preventing a human disorder or disease comprising, administering a therapeutically-effective amount of the pharmaceutical composition of the invention, such that therapy or prevention of the human disease or disorder is achieved.

In another aspect, the invention provides a method of enhancing the antigen-binding affinity of an antibody comprising:

(a) determining a three-dimensional representation of an antibody-antigen interface,

(b) generating amino acid mutations of the antibody at the interface,

(c) generating a conformational search space of the mutations of step (b),

(d) comparing the conformational search space of (b) against a library of rotamers and organizing those mutations that have allowable conformational space as a search tree,

(e) pruning the search tree using the Dead End Elimination algorithm followed by the A* algorithm,

(f) calculating the lowest total energy of the remaining mutants of (e) by applying a molecular mechanics algorithm to the remaining mutants of (e),

(g) calculating the change in the change of the free energy of binding of the antibodies resulting from the mutants having conformations with the lowest total energy from (f) to an antigen by using a molecular mechanics Poisson-Boltzmann surface area algorithm on the antibodies of (f) bound to antigens,

(h) using an algorithm to evaluate the stability of the antibodies of step (g) having a change in the change of the free energy of binding of less than zero,

(i) restricting the number of antibodies generated from step (h) to antibodies having amino acid mutations caused by at least two changes in the nucleic acid bases of codons for the amino acid mutations of the antibodies of step (h),

(j) using the amino acid mutations of the antibodies of step (i) to design a focused library of point mutations for in vitro affinity maturation,

(k) selecting an amino acid residue of the antibody from the library of point mutations of step(j) for substitution in the antibody such that upon substitution, the antigen-binding affinity of the antibody is enhanced.

In another aspect, the invention provides a method of identifying a variant of an enzyme with enhanced substrate binding affinity, the method comprising:

(a) determining a three-dimensional representation of an enzyme-substrate interface of a first enzyme,

(b) conformationally sampling point mutations of amino acid residues of the enzyme at the enzyme-substrate interface,

(c) selecting the point mutations of step (b) that are conformationally allowed,

(d) selecting the point mutations of step (c) that have both a Boltzmann averaged predictor based on a change of a change of a free energy of binding (ΔΔG*) of less than zero kcal/mol and a Boltzmann averaged predictor based on only a change of a change of the polar component of the free energy of binding (ΔΔEₚₒₗ*) of less than zero kcal/mol, and

(e) creating a library of enzymes containing at least one point mutation from the point mutations of step (d),

(f) screening the enzymes of step (e) for enhanced substrate-binding affinity in vitro; and

(g) selecting an enzyme of step (f) having enhanced substrate binding affinity relative to the first enzyme, thereby identifying a variant of an enzyme with enhanced substrate binding affinity.

In one embodiment, the amino acid residues of the enzyme at the enzyme-substrate interface that are conformationally sampled in step (b) are limited to those amino acid residues that are determined by in silico alanine screening to have an effect on the overall change in the change of free energy of binding of less than 1 kcal/mol.

In another embodiment, the amino acid residues of the enzyme at the enzyme-substrate interface that are conformationally sampled in step (b) are limited to those amino acid residues that are point mutations caused by at least two nucleic acid base changes in the codon coding for the point mutation of the enzyme.

In another embodiment, the amino acid residues of the enzyme at the enzyme-substrate interface that are conformationally sampled in step (b) are limited to those amino acid residues that are point mutations that do not cause a change in the stability of the modified, mutated or altered enzyme that is greater than 3 kcal/mol.

In another embodiment, the three-dimensional representation is a crystal structure having a resolution of about 2.5 Angstroms or less.

In another embodiment, the point mutations of the amino acid residues of step (b) comprise an alteration of the side chains of the amino acid residues.

In another embodiment, the method further comprises expressing the modified, mutated or altered enzyme.

In another embodiment, the method is repeated at least one time.

In another embodiment, at least one step is informed by data selected from the group consisting of substrate binding data derived from an expressed enzyme binding to a substrate in a solvent, crystal structure data of an enzyme, crystal structure data of an enzyme bound to a substrate, three-dimensional structural data of an enzyme, NMR structural data of an enzyme, and computer-modeled structural data of an enzyme.

In another embodiment, the modified enzyme is expressed in an expression system selected from the group consisting of an acellular extract expression system, a phage display expression system, a prokaryotic cell expression system, and a eukaryotic cell expression system.

In another embodiment, the enzyme is modified at one or more positions at the enzyme active site.

In another embodiment, the substrate binding affinity of the enzyme is predicted to be increased by a factor of about 1.1, 1.2, 1.3, 1.4, 1.5, 1.6, 1.7, 1.8, 1.9, 2, 3, 5, 8, 10, 50, 10², 10³, 10⁴, 10⁵, 10⁶, 10⁷, or 10⁸.

In another aspect, the invention provides a plurality of enzymes produced by the method of the invention.

In another aspect, the invention provides a nucleic acid encoding the enzyme.

In another aspect, the invention provides a host cell encoding the nucleic acid.

In another aspect, the invention provides an enzyme produced by culturing the host cell under conditions such that the enzyme is expressed.

In another aspect, the invention provides pharmaceutical composition comprising the enzyme.

In another aspect, the invention provides a method for treating or preventing a human disorder or disease comprising, administering a therapeutically-effective amount of the pharmaceutical composition, such that therapy or prevention of the human disease or disorder is achieved.

In one embodiment, the catalytic efficiency of the enzyme is increased through the point mutations of step (d).

In another embodiment, a generalized Born model is used instead of a Poisson Boltzmann model.

In another embodiment, the method further comprises modeling the contribution of crystallographic waters to the free energy of binding of the antibodies of step d to an antigen.

In another embodiment, the method further comprises modeling the antibody-antigen interface using a molecular dynamics algorithm to quantify the entropic part of the free energy of binding of the antibody to an antigen.

In another embodiment, the method further comprises calculating the free energy of binding with the additional contribution to the free energy of binding of the antibody to an antigen from modeling the backbone flexibility of the antibody.

### BRIEF DESCRIPTION OF THE FIGURES

FIG. 1: Effect of the cutoff on various parameters when using the double criterion (ΔΔG* & ΔΔEₚₒₗ*<cutoff) predictor. The success rate is the ratio of TP among the predicted positives, whereas the overall success rate is the ratio of true predictions and all predictors, i.e. (TP+TN)/(TP+TN+FP+FN).

FIG. 2: Comparison of (A) in vitro and (B) in silico (B) alanine scanning analysis. A and B pictures are representations of the antibody-antigen interface of anti-HER2 bH1, viewed from the antigen side. In the two cases the light chain is on the left, and the heavy chain is on the right. Residues that were analysed by alanine scanning are represented as surface. Residues predicted as hotspots by the in silico study are surrounded by a dashed line in the two representations. Residues selected by in vitro analysis to be randomized are symbolized by * and X (corresponding to positions where an enhancing mutation was found or not, respectively). A: residues found as hot-spot by in vitro alanine scanning were colored in Orange (0.5 kcal/mol<ΔΔG<1 kcal/mol) or red (ΔΔG>1 kcal/mol). B: residues predicted as hotspots by in silico alanine scanning are colored in yellow.

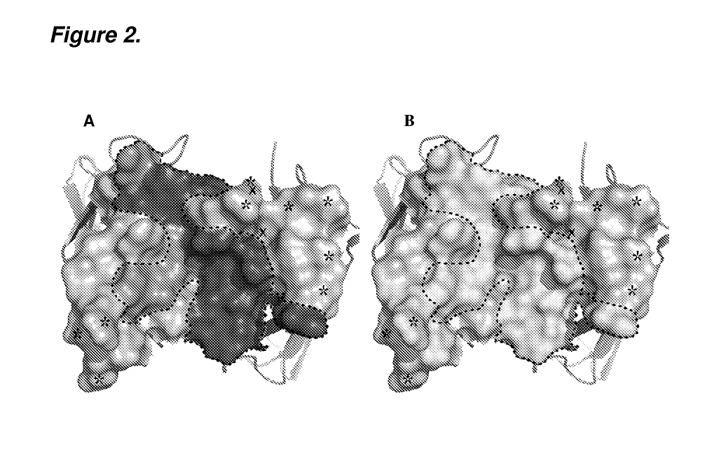

FIG. 3: In silico affinity maturation workflow. The starting point is a three dimensional structure of the protein-protein complex. Residues to mutate are selected by CDR definition or by proximity to the partner of interaction (interface residues). Subsequently, each mutation is processed separately. The conformational search space, limited to the side chains of mutated residues and to side chains of neighbouring residues, is generated by using a library of rotamers. The search space is organized as a search tree, Dead End Elimination pruning and the A* algorithm are applied. The resulting conformations with the lowest total energy (calculated with MM) are then submitted to MM/PBSA calculation in order to estimate the effect of the mutation on ΔΔG_{binding}. The stability of all mutations is verified by EGAD. The resulting dataset can be restricted by their Δcodon-base, in order to the lower the number of proposals. The outputs are the list of best mutations that could be used to propose point mutations. Alanine-scanning detection of hot-spots can be used to orient the design of a focused library for in vitro affinity maturation.

### DETAILED DESCRIPTION OF THE INVENTION

The implementation of a structure based virtual affinity maturation protocol and evaluation of its predictivity are presented herein. The in silico affinity maturation protocol is based on conformational sampling of the interface residues (using the DEE/A* algorithm), followed by the estimation of the change of free energy of binding due to a point mutation by applying MM/PBSA calculations. The protocol has been evaluated for 173 mutations in 7 different protein complexes for which experimental data were available. The use of the Boltzamnn averaged predictor based on the free energy of binding (ΔΔG*) combined with the one based on its polar component only (ΔΔEpol*) led to the proposal of a subset of mutations out of which 45% would have successfully enhanced the binding. When focusing on those mutations that are more difficult to be introduced by experimental methods (99 mutations with at least two base changes in the codon), the success rate is increased to 63%. In another evaluation, focusing on 56 Alanine scanning mutations, the in silico protocol was able to detect 89% of the hot spots. Accordingly, the methods presented herein are useful for guiding the in vitro affinity maturation of antibody and proteins.

The best predictor to find affinity enhancing mutations was found to be the double criteria predictor (ΔΔEₚₒₗ*<0 kcal/mol and ΔΔG*<0 kcal/mol), with a 45% success rate measured on 7 different systems totaling 173 point mutations. The number of mutations proposed by in silico affinity maturation can be further reduced with no impact on the success rate in different ways, as follows. For example, if the number of candidate mutations that is generated is too large to be handled experimentally, lowering the threshold up to −1 kcal/mol is an option. By considering only mutations involving more than 1 codon base change vs. the wild-type sequence, the number of mutations using systems that have been analyzed by other in vitro systems, such as phage display, will be reduced because those systems typically only evaluate amino acid point mutations caused by a change of one nucleic base in the codon encoding the point mutation. This criterion can be explicitly considered in the selection strategy and results in an increase in the success rate. In the particular subset of mutations provided herein, the increase in the success rate when considering point mutations caused by having more than one codon base change is from 45% to 63%.

In the particular case where alanine mutations only are considered, the structure-based virtual affinity maturation protocol presented in this study was highly predictive to identify the hot spots of interaction (in silico alanine scanning) The potential usefulness of such an in silico alanine scanning protocol has been demonstrated by applying it to an antibody-antigen complex, where in vitro randomization would have been focused on 19 optimizable positions out of 35 positions in contact with the antigen.

Two types of evaluation of variant antibodies for increased binding affinity presented herein illustrate how a structure-based virtual affinity maturation protocol can be applied to support two complementary antibody design strategies. The first strategy consists in identifying specific mutations, and proposes a limited set of mutants for further cloning, expression and characterization. The second strategy consists in identifying specific positions where experimental randomization can be performed.

Embodiments of the methods described herein can be used to obtain a variant binding protein having increased binding affinity for an antigen in comparison to a native antibody. Changes to the antibody are introduced according to a set of discrete criteria or rules as described herein. Candidate amino acid side chain positions, and residue modifications at these positions, are then determined based, for example, on the potential gain in binding free energy observed in the optimizations. As described herein, the designed variant antibodies can be built in silico and the binding energy recalculated. Accordingly, when the desired side chain chemistries are determined for the candidate amino acid position(s) according to the predictive protocols herein presented, the residue position(s) is then modified or altered, e.g., by substitution, insertion, or deletion, as further described herein. Results from these computational modification calculations may then reevaluated as needed, for example, after subsequent reiterations of the method either in silico or informed by additional experimental structural and functional data.

In terms of applications, the scope of the present disclosure goes beyond the optimization of monoclonal antibodies. Many other therapeutic proteins and even catalytic RNAs could benefit from this type of approach. All the antibody derived formats can be cited, as well as more recent formats based on other folds (e.g., fibronectin, lipocalin, ankyrin) and also peptides such as insulin. The in silico protocol presented herein can be useful to improve the binding affinity to a pathogenic antigen, and therefore aims at obtaining a better pharmacological effect in the patient. Cross-reactivity with the antigen in other species is another property of interest for which the protocol can be of use. In such a case, the protocol could be useful to obtain a protein that binds, with equivalent potency, to both the pathogenic antigen, e.g. human, and the antigen in a species relevant for toxicity studies, e.g. cynomologus.

### DEFINITIONS

In order to provide a clear understanding of the specification and claims, the following definitions are conveniently provided below.

The term “structure”, or “structural data”, as used herein, includes the known, predicted and/or modeled position(s) in three-dimensional space that are occupied by the atoms, molecules, compounds, amino acid residues and portions thereof, and macromolecules and portions thereof, of the disclosure, and, in particular, an antibody bound to an antigen in a solvent. A number of methods for identifying and/or predicting structure at the molecular/atomic level can be used such as X-ray crystallography, NMR structural modeling, and the like.

The term “binding affinity”, as used herein, includes the strength of a binding interaction and therefore includes both the actual binding affinity as well as the apparent binding affinity. The actual binding affinity is a ratio of the association rate over the disassociation rate. Therefore, conferring or optimizing binding affinity includes altering either or both of these components to achieve the desired level of binding affinity. The apparent affinity can include, for example, the avidity of the interaction. For example, a bivalent altered variable region binding fragment can exhibit altered or optimized binding affinity due to its valency. Binding affinities may also be modeled, with such modeling contributing to selection of residue alterations in the methods of the current disclosure.

The term “binding free energy” or “free energy of binding”, as used herein, includes its art-recognized meaning, and, in particular, as applied to antibody-antigen interactions in a solvent. Reductions in binding free energy enhance antibody-antigen affinities, whereas increases in binding free energy reduce antibody-antigen affinities.

The term “solvent”, as used herein, includes its broadest art-recognized meaning, referring to any liquid in which an antibody of the instant disclosure is dissolved and/or resides.

The term “energy landscape” as used herein includes an energy distribution where peaks and wells define ensemble states of a molecule. It is believed that an energy landscape can provide a complete description of the folding process as well as descriptions of local structural states, whereas the common optimized or minimized structure describes only a single structural species out of a collection of many possible states within a local energy minimum.

The term “lead sequence” as used herein includes the sequence used for searching sequence database.

The term “hit library” or “library” as used herein includes a collection of sequences found by searching the sequence database using the lead sequence or sequence profile.

The term “hit variant library” or “variant library” as used herein includes an in silico amino acid sequence library derived from the combinatorial enumeration of the variant profile of the hit library. A Hit variant library is an amino acid sequence library that is expressed in vitro by a degenerate oligonucleotide library (below) for functional screening. Hit variant libraries expand the sequence space of other hit variant libraries due to back translation, optimized codon usage, recombination at the nucleotide level and expression of the resulting combinatorial nucleic acid library.

The term “refined amino acid library” as used herein includes an in silico amino acid sequence library derived from a hit variant library as a result of a re-profiling or specific design. Re-profiling of the variants can be accomplished 1) by selecting a sequence cluster(s) based energy ranking with a specific cut off value or a window of sequences containing key amino acid residues, 2) by including specific positional residues identified by functional screening, and/or 3) by inclusion or exclusion of residues or sequence clusters as determined by those trained in the arts using any other means available for making such determinations.

The term “degenerate nucleic acid library” as used herein includes the library of mixed oligonucleotides that is used to target an amino acid variant profile that corresponds to a designed amino acid library. It is derived from the combinatorial enumeration of the corresponding nucleic acid positional variant profile that is back translated from the amino acid positional variant profile of libraries using optimized codon(s).

The term “combinatorial amino acid library” as used herein includes a library generated from the complete combinatorial enumeration of an amino acid positional variant profile.

The term “combinatorial nucleic acid library” as used herein includes a library generated from the complete combinatorial enumeration of a nucleic acid positional variant profile.

The term “DNA shuffling” as used herein refers to a method of generating recombinant oligonucleotides from a mixture of parental sequences through multiple iterations of oligonucleotide fragmentation and homologous recombination (Stemmer W P (1994) Nature 370, 389-391).

The term “in silico rational library design” as used herein refers to a method of designing a digital amino acid or nucleic acid library that incorporates evolutionary, structural, and functional data in order to define and efficiently sample ensembles in the sequence and structure spaces in order to identify those that have a desired fitness.

The term “side chain rotamer” as used herein refers to the conformation of an amino acid side chain defined in terms of the dihedral angels or chi angles of side chains.

The term “rotamer library” as used herein refers to a distribution of side chain rotamers either based on the backbone dihedral angles phi and psi called backbone-dependent rotamer library or independent of backbone dihedral angles called backbone-independent rotamer library for all amino acids derived from the analysis of side chain conformations in a protein structural database.

The term “antibody”, as used herein, includes monoclonal antibodies (including full length monoclonal antibodies), polyclonal antibodies, multispecific antibodies (e.g., bispecific antibodies), chimeric antibodies, CDR-grafted antibodies, humanized antibodies, human antibodies and antigen-binding fragments thereof, for example, an antibody light chain (VL), an antibody heavy chain (VH), a single chain antibody (scFv), a F(ab′)2 fragment, a Fab fragment, an Fd fragment, an Fv fragment, and a single domain antibody fragment (DAb).

The term “chimeric antibody” is used to describe a protein comprising at least an antigen-binding portion of an immunoglobulin molecule that is attached by, for example, a peptide bond or peptide linker, to a heterologous protein or a peptide thereof. The “heterologous” protein can be a non-immunoglobulin or a portion of an immunoglobulin of a different species, class or subclass.

The term “antigen”, as used herein, includes an entity (e.g., a proteinaceous entity or peptide) to which an antibody specifically binds, and includes, e.g., a predetermined antigen to which both a parent antibody and modified antibody as herein defined bind. The target antigen may be polypeptide, carbohydrate, nucleic acid, lipid, hapten, or other naturally occurring or synthetic compound. Preferably, the target antigen is a polypeptide.

The term “CDR”, as used herein, includes the complementarity determining regions as described by, for example Kabat, Chothia, or MacCallum et al., (see, e.g., Kabat et al, In “Sequences of Proteins of Immunological Interest,” U.S. Department of Health and Human Services, 1983; Chothia et al., J. Mol. Biol. 196:901-917, 1987; and MacCallum et al., J. Mol. Biol. 262:732-745 (1996); the contents of which are incorporated herein in their entirety).

The term “variable region”, as used herein, includes the amino terminal portion of an antibody which confers antigen binding onto the molecule and which is not the constant region. The term is intended to include functional fragments, for example, antigen-binding fragments, which maintain some or all of the binding function of the whole variable region.

The term “framework region”, as used herein, includes the antibody sequence that is between and separates the CDRs.

The term terms “modified”, “variant”, “mutant” or “altered”, as used herein, include antibodies or antigen-binding fragments thereof, that contain one or more amino acid changes in, for example, a CDR(s), a framework region(s), or both as compared to the parent amino acid sequence at the changed position. A modified or altered antibody typically has one or more residues which have been substituted with another amino acid residue, related side chain chemistry thereof, or one or more amino acid residue insertions or deletions.

The term “parent antibody”, “original antibody”, “starting antibody”, “wild-type”, “lead antibody”, “lead sequence” or “first antibody”, as used herein, includes any antibody for which modification of antibody-antigen binding affinity by the methods of the instant disclosure is desired. Thus, the parent antibody represents the input antibody on which the methods of the instant disclosure are performed. The parent polypeptide may comprise a native sequence (i.e. a naturally occurring) antibody (including a naturally occurring allelic variant), or an antibody with pre-existing amino acid sequence modifications (such as insertions, deletions and/or other alterations) of a naturally occurring sequence. The parent antibody may be a monoclonal, chimeric, CDR-grafted, humanized, or human antibody.

The terms “antibody variant”, “modified antibody”, “antibody containing a modified amino acid”, “mutant”, or “second antibody”, “third antibody”, etc., as used herein, include an antibody which has an amino acid sequence which differs from the amino acid sequence of a parent antibody. Preferably, the antibody variant comprises a heavy chain variable domain or a light chain variable domain having an amino acid sequence which is not found in nature. Such variants necessarily have less than 100% sequence identity or similarity with the parent antibody. In a preferred embodiment, the antibody variant will have an amino acid sequence from about 75% to less than 100% amino acid sequence identity or similarity with the amino acid sequence of either the heavy or light chain variable domain of the parent antibody. Typically, N-terminal, C-terminal, or internal extensions, deletions, or insertions into the antibody sequence outside of the variable domain are not construed as affecting sequence identity or similarity. The antibody variant is generally one which comprises one or more amino acid alterations in or adjacent to one or more hypervariable regions thereof. The modified antibodies of the present disclosure may be modeled in silico and/or expressed.

The phrase “candidate amino acid residue position” or “hot spot”, as used herein, includes an amino acid position identified within an antibody of the present disclosure, wherein the substitution of the candidate amino acid is modeled, predicted, or known to impact the binding affinity of the antibody upon alteration, deletion, insertion, or substitution with another amino acid.

The term “elected amino acid”, as used herein, refers to an amino acid residue(s) that has been selected by the methods of the present disclosure for substitution as a replacement amino acid at the candidate amino acid position within the antibody. Substitution of the candidate amino acid residue position with the elected amino acid residue may either reduce or increase the binding free energy of the antibody-antigen complex.

The terms “amino acid alteration”, “alteration for said amino acid”, “amino acid modification” or “point mutation”, as used herein, include refers to a change in the amino acid sequence of a predetermined amino acid sequence. Exemplary alterations include insertions, substitutions, and deletions. The terms “amino acid alteration”, “alteration for said amino acid”, “amino acid modification” or “point mutation” as used herein, includes the replacement of an existing amino acid residue side chain chemistry in a predetermined amino acid sequence with another different amino acid residue side chain chemistry, by, for example, amino acid substitution. Individual amino acid modifications of the instant disclosure are selected from any one of the following: (1) the set of amino acids with nonpolar sidechains, e.g., Ala, Cys, Ile, Leu, Met, Phe, Pro, Val, (2) the set of amino acids with negatively charged side chains, e.g., Asp, Glu, (3) the set of amino acids with positively charged sidechains, e.g., Arg, His, Lys, and (4) the set of amino acids with uncharged polar sidechains, e.g., Asn, Cys, Gln, Gly, His, Met, Phe, Ser, Thr, Trp, Tyr, to which are added Cys, Gly, Met and Phe.

The term “naturally occurring amino acid residue”, as used herein, includes one encoded by the genetic code, generally selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val).

The term “non-naturally occurring amino acid residue” as used herein, includes an amino acid residue other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) can be used. Briefly, these procedures involve chemically activating a suppressor tRNA with a non-naturally occurring amino acid residue followed by in vitro transcription and translation of the RNA.

The term “treatment” refers to both therapeutic treatment and prophylactic or preventative measures. Those in need of treatment include those already with the disorder as well as those in which the disorder is to be prevented.

The term “disorder or disease” is any condition that would benefit from treatment with the antibody variant. This includes chronic and acute disorders or diseases including those pathological conditions which predispose the mammal to the disorder in question.

The terms “cell”, “cell line”, “cell culture”, or “host cell”, as used herein, includes “transformants”, “transformed cells”, or “transfected cells” and progeny thereof. Host cells within the scope of the disclosure include prokaryotic cells such as E. coli, lower eukaryotic cells such as yeast cells, insect cells, and higher eukaryotic cells such as vertebrate cells, for example, mammalian cells, e.g., Chinese hamster ovary cells and NSO myeloma cells.

The details of one or more embodiments of the disclosure are set forth in the description below. Other features, objects, and advantages of the disclosure will be apparent from the detailed description and the claims.

### A. Obtaining a Parent Protein, e.g., Antibody or Antigen-Binding Fragment Thereof

The methods of the disclosure that are aimed at generating a non-naturally occurring binding protein (e.g., a non-naturally occurring antibody or an antigen-binding fragment thereof) can, but do not necessarily, begin by obtaining a binding protein. That protein may be referred to herein as a “parent” binding protein or sometimes as a “first” binding protein, and it can be used to obtain information that will allow one to modify or alter one or more amino acid residues either within that protein (i.e., within the parent antibody) or within a modified or altered protein having a sequence that is similar to, or that contains portions of, the sequence of the parent protein. As described herein, for example, one or more of the CDRs (or portions thereof) of a parent antibody, can be replaced with the corresponding CDR(s) of the modified antibody by standard genetic engineering techniques to accomplish the so-called CDR graft or transplant. Accordingly, the method can begin with a mammalian monoclonal or polyclonal antibody (e.g., murine or primate), chimeric, CDR-grafted, humanized, or human antibody.

Parent antibodies can be obtained from art-recognized sources or produced according to art-recognized technologies. For example, the parent antibody can be a CDR-grafted or humanized antibody having CDR regions derived from another source or species, e.g., murine. The parent antibody or any of the modified antibodies of the disclosure can be in the format of a monoclonal antibody. Methods for producing monoclonal antibodies are known in the art (see, e.g., Kohler and Milstein, Nature 256:495-497, 1975), as well as techniques for stably introducing immunoglobulin-encoding DNA into myeloma cells (see, e.g., Oi et al., Proc. Natl. Acad. Sci. USA 80:825-829, 1983; Neuberger, EMBO J. 2:1373-1378, 1983; and Ochi et al., Proc. Natl. Acad. Sci. USA 80:6351-6355, 1983). These techniques, which include in vitro mutagenesis and DNA transfection, allow for the construction of recombinant immunoglobulins; these techniques can be used to produce the parent and modified antibodies used in the methods of the disclosure or to produce the modified antibodies that result from those methods. Alternatively, the parent antibodies can be obtained from a commercial supplier. Antibody fragments (scFvs and Fabs) can also be produced in E. coli. The parent antibody or any of the modified antibodies of the disclosure can be an antibody of the IgA, IgD, IgE, IgG, or IgM class.

In certain embodiments, the methods of the disclosure can be used to alter and optimize the parent binding protein to generate a modified binding protein with improved binding affinity. In the methods of the disclosure, the parent and modified antibodies can be of the same or of different species (e.g., the parent antibody can be a non-human antibody (e.g., a murine antibody), and the modified antibody can be a human antibody). The antibodies can also be of the same, or of different, classes or subclasses. Regardless of their origin or class, portions of the sequences of the two antibodies can be identical to one another. For example, the FRs of the parent antibody can be identical to the FRs of the modified antibody. This would occur, for example, where the parent antibody is a human antibody and the modified antibody varies from the parent antibody only in that the modified antibody contains one or more non-human CDRs (i.e., in the modified antibody, one or more of the original, human CDRs have been replaced with a non-human (e.g., murine) CDR).

The methods of the disclosure can be carried out with parental antibodies that have the structure of a naturally occurring antibody. For example, the methods of the disclosure can be carried out with antibodies that have the structure of an IgG molecule (two full-length heavy chains and two full-length light chains). Thus, in some embodiments, the parent and/or modified antibody can include an Fc region of an antibody (e.g., the Fc region of a human antibody). The methods of the disclosure can be carried out, however, with less than complete antibodies; they can be carried out with any antigen-binding fragment of an antibody including those described further below (Fab fragments, F(ab′)₂ fragments, or single-chain antibodies (scFv)). The “fragments” can constitute minor variations of naturally occurring antibodies. For example, an antibody fragment can include all but a few of the amino acid residues of a “complete” antibody (e.g., the FR of V_{H} or V_{L} can be truncated). The fragments can be recombinantly produced and engineered, synthesized, or produced by digesting an antibody with a proteolytic enzyme. For example, the fragment can be an Fab fragment; digestion with papain breaks the antibody at the region, before the inter-chain disulphide bond, that joins the two heavy chains. This results in the formation of two identical fragments that contain the light chain and the V_{H} and C_{HI} domains of the heavy chain. Alternatively, the fragment can be an F(ab′)₂ fragment. These fragments can be created by digesting an antibody with pepsin, which cleaves the heavy chain after the inter-chain disulphide bond, and results in a fragment that contains both antigen-binding sites. Yet another alternative is to use a “single chain” antibody. Single-chain Fv (scFv) fragments can be constructed in a variety of ways. For example, the C-terminus of V_{H} can be linked to the N-terminus of V_{L}. Typically, a linker (e.g., (GGGGS)₄) is placed between V_{H} and V_{L}. However, the order in which the chains can be linked can be reversed, and tags that facilitate detection or purification (e.g., Myc-, His-, or FLAG-tags) can be included (tags such as these can be appended to any antibody or antibody fragment of the disclosure. Accordingly, and as noted below, tagged antibodies are within the scope of the present disclosure. In alternative embodiments, the antibodies used in the methods described herein, or generated by those methods, can be heavy chain dimers or light chain dimers. Still further, an antibody light or heavy chain, or portions thereof, for example, a single domain antibody (DAb), can be used.

Regardless of whether the method is carried out with a complete antibody or a fragment thereof, where all or part of the FR is present, the sequence of that FR can be that of a wild-type antibody. Alternatively, the FR can contain a mutation. For example, the methods of the disclosure can be carried out with a parent antibody that includes a framework region (e.g., a human FR) that contains one or more amino acid residues that differ from the corresponding residue(s) in the wild-type FR. The mutation can be one that changes an amino acid residue to the corresponding residue in an antibody of another species. Thus, an otherwise human FR can contain a murine residue (such mutations are referred to in the art as “back mutations”). For example, framework regions of a human antibody can be “back-mutated” to the amino acid residue at the same position in a non-human antibody. Such a back-mutated antibody can be used in the present methods as the “parent” antibody, in which case the “modified” antibody can include completely human FRs. Mutations in the FRs can occur within any of FR1, FR2, FR3, and/or FR4 in either V_{H} or V_{L} (or in V_{H} and V_{L}). Multiple residues in FR1, FR2, FR3, and/or FR4 can be changed from the naturally occurring residue (e.g., the human residue) to another residue (e.g., a donor residue, for example, murine residue, at the corresponding position)). The residues that immediately flank the CDRs are among those that can be mutated.

As the methods of the disclosure can be iterative, the parent antibody may not necessarily be a naturally occurring antibody or a fragment thereof. As the process of modifying an antibody can be repeated as many times as necessary, the starting antibody (or antigen-binding fragment thereof) can be wholly non-human or an antibody containing human FRs and non-human (e.g., murine) CDRs. That is, the “parent” antibody can be a CDR-grafted antibody that is subjected to the methods of the disclosure in order to improve the affinity of the antibody, i.e., affinity mature the antibody. In certain embodiments, the affinity may only be improved to the extent that it is about the same as (or not significantly worse than) the affinity of the naturally occurring human antibody (the FR-donor) for its antigen. Thus, the “parent” antibody may, instead, be an antibody created by one or more earlier rounds of modification, including an antibody that contains sequences of more than one species (e.g., human FRs and non-human CDRs). The methods of the disclosure encompass the use of a “parent” antibody that includes one or more CDRs from a non-human (e.g., murine) antibody and the FRs of a human antibody. Alternatively, the parent antibody can be completely human.

### B. Antibody-Antigen Structural Data

Proteins are known to fold into three-dimensional structures that are dictated by the sequences of their amino acids and by the solvent in which a given protein (or protein-containing complex) is provided. The three-dimensional structure of a protein influences its biological activity and stability, and that structure can be determined or predicted in a number of ways. Generally, empirical methods use physical biochemical analysis. Alternatively, tertiary structure can be predicted using model building of three-dimensional structures of one or more homologous proteins (or protein complexes) that have a known three-dimensional structure. X-ray crystallography is perhaps the best-known way of determining protein structure (accordingly, the term “crystal structure” may be used in place of the term “structure”), but estimates can also be made using circular dichroism, light scattering, or by measuring the absorption and emission of radiant energy. Other useful techniques include neutron diffraction and nuclear magnetic resonance spectroscopy (NMR). All of these methods are known to those of ordinary skill in the art, and they have been well described in standard textbooks (see, e.g., Physical Chemistry, 4th Ed., W. J. Moore, Prentiss-Hall, N.J., 1972, or Physical Biochemistry, K. E. Van Holde, Prentiss-Hall, N.J., 1971)) and numerous publications. Any of these techniques can be carried out to determine the structure of an antibody, or antibody-antigen-containing complex, which can then be analyzed according to the methods of the present disclosure and, e.g., used to inform one or more steps of the method of the disclosure. Similarly, these and like methods can be used to obtain the structure of an antigen bound to an antibody fragment, including a fragment consisting of, e.g., a single-chain antibody or Fab fragment. Methods for forming crystals of an antibody, an antibody fragment, or scFv-antigen complex have been reported by, for example, van den Elsen et al. (Proc. Natl. Acad. Sci. USA 96:13679-13684, 1999, which is expressly incorporated by reference herein).

Computational analysis using methods of the current disclosure are preferably applied to three dimensional models with a resolution of about less than 2 Å. However, methods of the current disclosure are useful for any three dimensional structural model of an antibody or binding protein.

The crystallographic structures of antibody/antigen complexes and of one non-immune-related protein/protein complex may be used to assemble a dataset upon which to test the results of predictions obtained from the methods of the present disclosure. Exemplary antibody/antigen complexes can be obtained from the Protein Data Bank (www.rcsb.org) and include the anti-lysosymes D44.1 (pdb code: 1MLC, resolution of 2.5 Å), D1.3 (pdb code: 1VFB, resolution of 1.8 Å) and HyHEL-63 (pdb code: 1DQJ, resolution of 2.0 Å), the anti-EGFR Cetuximab antibody (pdb code: 1YY9, resolution of 2.61 Å), the anti-HER2 (Herceptin) variant bH1 (pdb code: 3BE1, resolution of 2.9 Å), the anti-VLA1 antibody in complex with the I domain of the integrin VLA1 (pdb code: 1MHP, resolution of 2.8 Å), and a non-immune-related complex between barnase and barstar (pdb code: 1BRS, resolution of 2.0 Å).

In exemplary embodiments, protein complex models are altered in silico (e.g., using MOE software (Chemical Computing Group MOE (Molecular Operating Environment), 2009.10; Montreal, Canada, 2009) to have one or more of the following alterations: (1) Water molecules are deleted; (2) Missing residues are omitted from the model; (3) hydrogens are added. The resulting complex structures may be further minimized with harmonic constraints on all heavy atoms (100 kcal/mol/Å²) to remove steric clashes. For example, a AMBER94 force field may be used, with a reaction field model for implicit solvation, a cutoff of 10A on non-bonded interactions, a dielectric constant of 4 inside the protein, and 80 for the solvent. The protonate three dimensional protocol from MOE (Labute, P., Protonatethree dimensional: assignment of ionization states and hydrogen coordinates to macromolecular structures. Proteins 2009, 75, (1), 187-205) may be used to determine the tautomeric form and protonation state of histidines.

### C. Computational Analysis

As noted above, parental binding proteins are altered (or “modified”) according to the results of a computational analysis of forces between the binding protein and the protein to which it binds, preferably, in accordance to the discrete criteria or rules of the disclosure described herein. The computational analysis allows one to predict amino acid residues at or near the interface between the binding protein and its target (e.g., target antigen) and the target that can be mutated in order to improve the binding affinity of the binding protein for its target. The computational analysis can be mediated by a computer-implemented process. The computer program is adapted herein to consider the real world context of the binding interaction. The process is used to identify modifications to the structure of the binding protein that will increase the affinity between the modified binding protein and its target (compared to that of the unmodified (“starting” or “parent”) antibody. As is typical, the computer system (or device(s)) that performs the operations described here will include an output device that displays information to a user (e.g., a CRT display, an LCD, a printer, a communication device such as a modem, audio output, and the like). In addition, instructions for carrying out the method, in part or in whole, can be conferred to a medium suitable for use in an electronic device for carrying out the instructions. Thus, the methods of the disclosure are amendable to a high throughput approach comprising software (e.g., computer-readable instructions) and hardware (e.g., computers, robotics, and chips). The computer-implemented process is not limited to a particular computer platform, particular processor, or particular high-level programming language.

Starting with the three dimensional structure between an antigen and its antibody is very useful to focus the antibody library towards sequences with good probability of binding the antigen. In the presence of the antigen, the complete thermodynamic cycle of complex formation between an antibody and an antigen may be included in the calculation. The conformation of the antibody, especially in the combining site, may be modeled based on individual CDR loop conformation from its canonical family with preferred side-chain rotamers as well as the interactions between CDR loops. A wide range of conformations, including those of the side chains of amino acid residues and those of the CDR loops in the antigen combining site, can be sampled and incorporated into a main framework of an antibody. With the antigen present, such conformational modeling assures higher physical relevancy in the scoring, using physical-chemical force fields as well as semi-empirical and knowledge-based parameters, and better representation of the natural process of antibody, production and maturation in the body.

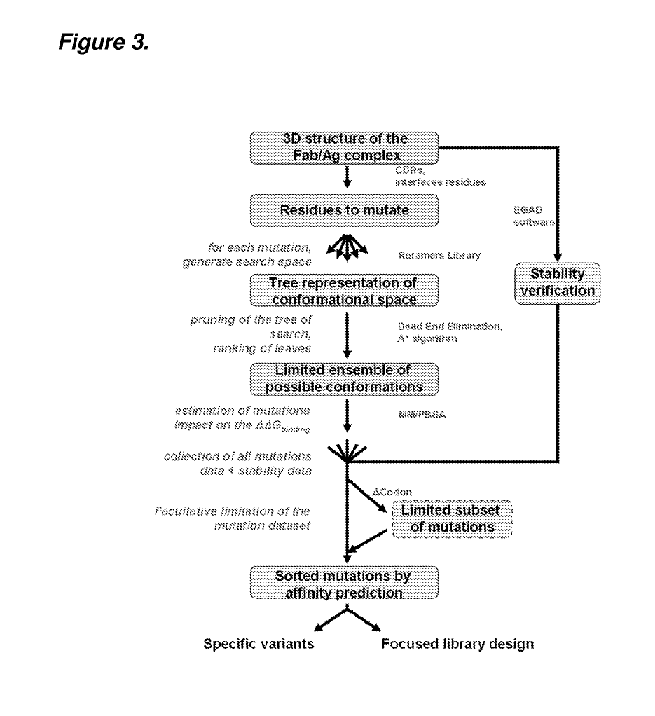

As illustrated in FIG. 3, the in silico affinity maturation method of the invention may include different computational steps, which deal mainly with conformational sampling and calculation of the free energy of binding. In general, in silico affinity maturation is a two-step process: the first step is to generate a set of mutations in silico using a conformational sampling algorithm, e.g., Dead End Elimination (DEE) algorithm and/or an A* algorithm. The second step is the calculation of the free energy of binding between a binding protein having one or more of the candidate mutations and the antigen. The various steps of the protocol (e.g., conformational sampling, estimation of binding energy, estimation of internal energy or stability) can be performed with art-recognized modelling software including, for example, MOE, CHARMM, AMBER, ROSETTA, EGAD and the like.

In an embodiment, the first step of the computational processing is done by calculating two sets of interactions for each rotamer at every position: the interaction of the rotamer side chain with the template or backbone, and the interaction of the rotamer side chain with all other possible rotamers at every other position, whether that position is varied or floated. It should be understood that the backbone in this case includes both the atoms of the antibody structure backbone, as well as the atoms of any fixed residues, wherein the fixed residues are defined as a particular conformation of an amino acid.

Once the energies of the variant antibodies are calculated and stored, the next step of the computational processing may occur. Preferred embodiments utilize a Dead End Elimination (DEE) followed by an A* algorithm step, and then preferably applying the Poisson-Boltzmann equation to evaluate the electrostatics profile of a variant.

Two sets of interactions are then calculated for each rotamer at every position: the interaction of the rotamer side chain with all or part of the backbone, and the interaction of the rotamer side chain with all other possible rotamers at every other position or a subset of the other positions. The energy of each of these interactions is calculated through the use of a variety of scoring functions, which include the energy of van der Waal's forces, the energy of hydrogen bonding, the energy of secondary structure propensity, the energy of surface area solvation and the electrostatics. Thus, the total energy of each rotamer interaction, both with the backbone and other rotamers, is calculated, and stored in a matrix form.

The discrete nature of rotamer sets allows a simple calculation of the number of rotamer sequences to be tested. A backbone of length n with m possible rotamers per position will have mⁿ possible rotamer sequences, a number which grows exponentially with sequence length and renders the calculations either unwieldy or impossible in real time. Accordingly, to solve this combinatorial search problem, a DEE calculation is performed followed by a A* calculation. The DEE calculation is based on the fact that if the worst total interaction of a first rotamer is still better than the best total interaction of a second rotamer, then the second rotamer cannot be part of the global optimum solution. Since the energies of all rotamers have already been calculated, the DEE approach only requires sums over the sequence length to test and eliminate rotamers, which speeds up the calculations considerably. DEE can be rerun comparing pairs of rotamers, or combinations of rotamers, which will eventually result in the determination of a single sequence which represents the global optimum energy.

Once the global solution has been found, a molecular mechanics/Poisson-Boltzmann sampling may be done to generate a rank-ordered list of sequences in the neighborhood of the DEE solution. Starting at the DEE solution, random positions are changed to other rotamers, and the new sequence energy is calculated.

In an alternate embodiment, only some of the residue positions of the antibody are variable, and the remainder are “fixed”, that is, they are identified in the three dimensional structure as being in a set conformation. In some embodiments, a fixed position is left in its original conformation (which may or may not correlate to a specific rotamer of the rotamer library being used). Alternatively, residues may be fixed as a non-wild type residue; for example, when known site-directed mutagenesis techniques have shown that a particular residue is desirable, the residue may be fixed as a particular amino acid. Alternatively, the methods of the present disclosure may be used to evaluate mutations de novo, as is discussed below. In an alternate preferred embodiment, a fixed position may be “floated”; the amino acid at that position is fixed, but different rotamers of that amino acid are tested. In this embodiment, the variable residues may be at least one, or anywhere from 0.1% to 99.9% of the total number of residues. Thus, for example, it may be possible to change only a few (or one) residues, or most of the residues, with all possibilities in between.

In one embodiment of the present disclosure, computational antibody modeling and design can be used for library design. In some embodiments, the reference antibody has a known three dimensional structure (e.g., there are three dimensional coordinates for each atom of the reference antibody) which can be used to generate a scaffold antibody. Generally this can be determined using X-ray crystallographic techniques, NMR techniques, de novo modeling, homology modeling, etc. Based on the three dimensional coordinates for each atom, optimal variants (e.g., having substantially similar coordinates and/or global energy) can be calculated.

Once an antibody structure backbone is generated (with alterations, as outlined above) and input into the computer, explicit hydrogens are added if not included within the structure (for example, if the structure was generated by X-ray crystallography, hydrogens must be added). After hydrogen addition, energy minimization of the structure is run, to relax the hydrogens as well as the other atoms, bond angles and bond lengths.

(1) Conformational Search Space

To limit the search space to a reasonable size, the backbone may be fixed and a search carried out using a limited number of side-chain rotamers. In certain embodiments, the DeMaeyer library may be employed (De Maeyer, M., et al., “All in one: a highly detailed rotamer library improves both accuracy and speed in the modelling of sidechains by dead-end elimination”. Fold Des 1997, 2, (1), 53-66, incorporated by reference herein). The De Maeyer library provides a good compromise between a thorough sampling of three dimensional conformations and ensuring the convergence of the conformational sampling by the DEE-A* algorithm. Hydroxyl and sulfhydryl sampling may be included (e.g., every 30°) to the library, in order to allow a conformational sampling with an all atom model. All neighbouring side chains with at least one heavy atom within a radius of 4 Å of the heavy atoms of the side chain of the mutated residue may also considered as flexible, and their degrees of freedom sampled together with the mutated residue.

(2) Conformational Sampling Algorithm (DEE/A*)

As described in the work of Desmet et al. (Desmet, J. M., Marc De; Hazes, B.; Lasters, I., The dead-end elimination theorem and its use in protein side-chain positioning. Nature 1992, 356, (6369), 539-542, incorporated by reference herein), the conformational search space can be considered as a search tree, by exploiting the pairwise decomposability of the interactions in the physical model. Therefore, the first step of the analysis consists of a calculation of an energy matrix of pairwise interactions. This step can be carried out with the CHARMm software (Brooks, B. R. et al., CHARMM: the biomolecular simulation program. J Comput Chem 2009, 30, (10), 1545-614). A short minimization may be performed with the CHARMM27 force field (MacKerell, A. D., Jr.; et al., Development and current status of the CHARMM force field for nucleic acids. Biopolymers 2000, 56, (4), 257-65). One search tree may be built for each mutation. The Dead-End Elimination (DEE) may be used to limit the size of the tree (see Desmet, J. M., The dead-end elimination theorem and its use in protein side-chain positioning. Nature 1992, 356, (6369), 539-542; Goldstein R. F., Efficient rotamer elimination applied to protein side-chains and related spin glasses. Biophys J 1994, 66, (5), 1335-40, incorporated by reference herein), and the A* algorithm (Leach, A. R; Exploring the conformational space of protein side chains using dead-end elimination and the A* algorithm. Proteins 1998, 33, (2), 227-39, incorporated by reference herein) may be used to find the conformations with the lowest energies, including the global minimum energy conformation (GMEC). In certain embodiments, only the GMEC and conformations within 10 kcal/mol from the GMEC are kept, with a limit of the 30 lowest energy conformations per mutation.

Usually, a large number of conformations have to be evaluated, and algorithms such as MM/PBSA (molecular mechanics Poisson-Boltzmann surface area) is a useful evaluative tool that offers a good compromise between desired accuracy and time (Kollman, et al., “Calculating structures and free energies of complex molecules: combining molecular mechanics and continuum models”. Acc Chem Res 2000, 33, (12), 889-97, incorporated by reference herein).

(3) Computational Estimation of the Free Energy of Binding

For each mutation, the conformations obtained by the procedure described above are then submitted to a more accurate protocol using the AMBER software (Case, D. A., et al., “The Amber biomolecular simulation programs”. J Comput Chem 2005, 26, (16), 1668-88). Here the effect of a mutation on ΔG_{binding} is estimated by the difference between the energy of binding of the mutated system (ΔG_{binding,mut}) and the energy of binding of the reference system (ΔG_{binding,wt}):

ΔΔG_{binding}=ΔG_{binding,mut}−ΔG_{binding,wt} Equation 1

In general²¹, the free energy of binding can be decomposed into an enthalpic component in the gas phase (ΔH^{gas}_{binding}), a desolvation term (ΔG_{desolv}) to take solvation effects into account, and an entropic term (ΔS),

ΔG_{binding}=ΔH_{binding}^{gas}+ΔG_{desolv}−TΔS Equation 2

ΔH^{gas}_{binding} is the sum of the electrostatic (ΔH_{elec}) and van der Waals (ΔH_{vdW}) interactions between partners of the complex, and it includes also an internal energy term (ΔHᵢₙₜᵣₐ) in order to account for the structural changes induced by binding. For the purpose of calculating ΔΔG_{binding} an assumption can be made that the mutation will not significantly perturb the protein structures, therefore ΔHᵢₙₜᵣₐ=0 and subsequently ΔΔHᵢₙₜᵣₐ=0. The term ΔG_{desolv} is divided into a nonpolar (ΔG_{nonpol desolv}) and an electrostatic component (ΔG_{elec desolv}). The first term was calculated as the sum of a cavitation term and a solvent-solute van der Waals interaction term, the second term was evaluated using the Poisson Boltzmann equation. Given that the backbone had been kept fixed, and in order to allow for a large number of mutations to be evaluated, the impact of the mutations on translational, conformational and vibrational entropies was neglected (ΔΔS=0).

The Poisson-Boltzmann continuum electrostatic solvation model³⁴ and a continuum solvent van der Waals model³⁵ were used to calculate the ΔG_{desolv} term. The main parameters were as follows: Amber94 force field, a dielectric constant of 4 within the protein and 80 in the implicit solvent, ionic strength of 0.145 M, probe sphere of 1.4 A radius, grid size of 0.5 Å. ΔH^{gas}_{binding} was calculated with the same forcefield and parameters. Instead of using a single value from Poisson-Boltzmann calculations, the ΔG_{elec desolv} of each conformation was obtained by averaging the energies over three 0.125 Å translations along the xyz diagonal. This later step was performed to remove the dependency of the calculated value on the initial three dimensional position of the grid.

(4) Boltzmann Averaging of Energies

According to the protocol described above, each mutation results in a plurality (e.g., up to 30) conformations with the corresponding energy values. In certain embodiments, the energies are combined via a Boltzmann averaging procedure. Boltzmann averaging may be carried out in two different ways. First, by Boltzmann averaging of the ΔG_{binding} values of each complex, implying that for a mutation with multiple conformations i, Equation 3 can be written as follows:

$$Δ {G}_{binding}^{averaged} = {\sum }_{i} Δ {G}_{binding}^{i} \times \frac{{e}^{- Δ {G}_{total}^{i} / {k}_{B} T}}{{\sum }_{i} {e}^{- Δ {G}_{total}^{i} / {k}_{B} T}} \qquad Equation 3$$

Boltzmann averaging of the energies of the antigen A, of the antibody B, and of the complex AB before the calculation of an averaged ΔΔG_{binding}, as shown in Equation 4.

ΔG_{binding}^{averaged}=G_{AB}^{averaged}−G_{A}^{averaged}−G_{B}^{averaged} Equation 4

Each of the three terms the Boltzmann averaging scheme may be applied individually, see Equation 5.

$${G}_{x}^{averaged} = {\sum }_{i} {G}_{x}^{i ( x )} \times \frac{{e}^{- {G}_{total}^{i ( x )} / {k}_{B} T}}{{\sum }_{i} {e}^{- Δ {G}_{total}^{i ( x )} / {k}_{B} T}} \qquad Equation 5$$

(5) Computational Estimation of the Protein Stability

In certain embodiments, the effect of a mutation on protein stability may be evaluated or predicted. For example, the EGAD software (Tan, C., et al., Implicit nonpolar solvent models. J Phys Chem B 2007, 111, (42), 12263-74, incorporated by reference herein) may be used to predict the effect of the mutation on protein stability. It has been shown that this algorithm is able to estimate the stability of a protein with an average unsigned error of 1 kcal/mol (Pokala, N.; et al., Energy functions for protein design: adjustment with protein-protein complex affinities, models for the unfolded state, and negative design of solubility and specificity. J Mol Biol 2005, 347, (1), 203-27). Two indicators resulting from EGAD analysis may be used to predict stability. The first one is the folding energy variation due to the mutation. The second one is a confidence indicator based on the number of steric clashes with the environment induced by the mutation compared to the wild-type structure. If the surrounding is expected to be disturbed by the introduced mutation it is assumed to be a destabilizing mutation. If the EGAD estimation of stability change is higher than 3 kcal/mol or if the change in the number of clashes is higher than 2, then the mutation may be considered as destabilizing.

(6) Statistical Evaluation of Predictions

In certain embodiments, sensitivity and specificity scores may be used to assess the rate of positives or negatives found. The sensitivity is the total of true positives divided by the sum of true positives and false negatives. The specificity is the total of true negatives divided by the sum of true negatives and false positives. A value of 0.5 corresponds to a random prediction, and a lower value means a prediction worse than random. For example, the definition of success rate (SR), may be introduced as follows:

$$SR = \frac{TP}{TP + FP} \qquad Equation 6$$

TP is the number of true positives, and FP is the number of false positives.

In other embodiments, e.g., in the case of alanine scanning, the success rate may be based on the prediction of unfavourable mutation as follows:

$$SR = \frac{TN}{TN + FN} \qquad Equation 7$$

TN is the number of true negatives, and FN is the number of false negatives.

(7) Choice of a Predictor

Table 1 depicts the evaluation of six predictors regarding the impact of mutations on the ΔΔG_{binding}. The evaluation was performed for each protein system separately, as well as for all the mutations across all the systems (All). The predictors flagged by a * and ** have been Boltzmann-averaged according to Equation 3 and Equation 4 respectively. top ΔΔEₚₒₗ and top ΔΔG are non-averaged predictors based on the best conformation. Abbreviations used are as follows: TP (true positives), FP (false positives), TN (true negatives), FN (false negatives). Numbers that are not in bold refer to the number of TP, FP, TN or FN.

The predictions based on ΔΔEₚₒₗ*<0 kcal/mol or on ΔΔG*<0 kcal/mol showed a big overlap of true positives lists, but significant differences regarding the false positives. The combination of both (herein, the double criterion predictor) led to a better performance on false positives. With this more stringent criterion, a mutation will be predicted as enhancing the affinity if it improves both the total energy and its polar component. The result was a decrease of the number of false positive by 24%, leading to an increase of the success rate (from 40 to 45%) and of the specificity (from 72 to 79%). This double criterion was found to be best predictor for 173 mutations of the 7 systems (Table 1).

TABLE 1

| | | Exp. | | | | | | |
|---|---|---|---|---|---|---|---|---|
| | | (threshold = | | | | | | ΔΔG* & |
| | System | 0 kcal/mol) | ΔΔG* | ΔΔG** | ΔΔEpol* | top ΔΔG | top ΔΔEpol | ΔΔEpol* |
| TP | D44.1 | 6 | 5 | 4 | 5 | 3 | 4 | 5 |
| FP | | | 4 | 5 | 5 | 3 | 4 | 4 |
| TN | | 5 | 1 | 0 | 0 | 2 | 1 | 1 |
| FN | | | 1 | 2 | 1 | 3 | 2 | 1 |
| Sensitivity | | | 0.83 | 0.67 | 0.83 | 0.50 | 0.67 | 0.83 |
| Specificity | | | 0.20 | 0.00 | 0.00 | 0.40 | 0.20 | 0.20 |
| Success rate | | | 0.56 | 0.44 | 0.50 | 0.50 | 0.50 | 0.56 |
| TP | D1.3 | 3 | 1 | 1 | 2 | 1 | 2 | 1 |
| FP | | | 2 | 1 | 0 | 1 | 0 | 0 |
| TN | | 17 | 15 | 16 | 17 | 16 | 17 | 17 |
| FN | | | 2 | 2 | 1 | 2 | 1 | 2 |
| Sensitivity | | | 0.33 | 0.33 | 0.67 | 0.33 | 0.67 | 0.33 |
| Specificity | | | 0.88 | 0.94 | 1.00 | 0.94 | 1.00 | 1.00 |
| Success rate | | | 0.33 | 0.50 | 1.00 | 0.50 | 1.00 | 1.00 |
| TP | Cetuximab | 5 | 4 | 4 | 4 | 3 | 5 | 4 |
| FP | | | 0 | 0 | 0 | 0 | 0 | 0 |
| TN | | 0 | 0 | 0 | 0 | 0 | 0 | 0 |
| FN | | | 1 | 1 | 1 | 2 | 0 | 1 |
| Sensitivity | | | 0.80 | 0.80 | 0.80 | 0.60 | 1.00 | 0.80 |
| Specificity | | | 0.0 | 0.0 | 0.0 | 0.0 | 0.0 | 0.0 |
| Success rate | | | 1.00 | 1.00 | 1.00 | 1.00 | 1.00 | 1.00 |
| TP | Anti-VLA1 | 10 | 7 | 7 | 7 | 8 | 7 | 7 |
| FP | | | 19 | 17 | 15 | 18 | 17 | 13 |
| TN | | 57 | 38 | 40 | 42 | 39 | 40 | 44 |
| FN | | | 3 | 3 | 3 | 2 | 3 | 3 |
| Sensitivity | | | 0.70 | 0.70 | 0.70 | 0.80 | 0.70 | 0.70 |
| Specificity | | | 0.67 | 0.70 | 0.74 | 0.68 | 0.70 | 0.77 |
| Success rate | | | 0.27 | 0.29 | 0.32 | 0.31 | 0.29 | 0.35 |
| TP | HyHEL-63 | 0 | 0 | 0 | 0 | 0 | 0 | 0 |
| FP | | | 1 | 1 | 2 | 1 | 3 | 1 |
| TN | | 21 | 20 | 20 | 19 | 20 | 18 | 20 |
| FN | | | 0 | 0 | 0 | 0 | 0 | 0 |
| Sensitivity | | | 0 | 0 | 0 | 0 | 0 | 0 |
| Specificity | | | 0.95 | 0.95 | 0.90 | 0.95 | 0.86 | 0.95 |
| Success rate | | | 0.00 | 0.00 | 0.00 | 0.00 | 0.00 | 0.00 |
| TP | anti-HER2 | 11 | 7 | 7 | 7 | 5 | 5 | 7 |
| FP | | | 8 | 7 | 11 | 6 | 10 | 7 |
| TN | | 24 | 16 | 17 | 13 | 18 | 14 | 17 |
| FN | | | 4 | 4 | 4 | 6 | 6 | 4 |
| Sensitivity | | | 0.64 | 0.64 | 0.64 | 0.45 | 0.45 | 0.64 |
| Specificity | | | 0.67 | 0.71 | 0.54 | 0.75 | 0.58 | 0.71 |
| Success rate | | | 0.47 | 0.50 | 0.39 | 0.45 | 0.33 | 0.50 |
| TP | Barnase-Barstar | 1 | 0 | 0 | 0 | 0 | 0 | 0 |
| FP | | | 4 | 4 | 5 | 4 | 5 | 4 |
| TN | | 13 | 9 | 9 | 8 | 9 | 8 | 9 |
| FN | | | 1 | 1 | 1 | 1 | 1 | 1 |
| Sensitivity | | | 0.00 | 0.00 | 0.00 | 0.00 | 0.00 | 0.00 |
| Specificity | | | 0.69 | 0.69 | 0.62 | 0.69 | 0.62 | 0.69 |
| Success rate | | | 0.00 | 0.00 | 0.00 | 0.00 | 0.00 | 0.00 |
| TP | All | 36 | 24 | 23 | 25 | 20 | 23 | 24 |
| FP | | | 38 | 35 | 38 | 33 | 39 | 29 |
| TN | | 137 | 99 | 102 | 99 | 104 | 98 | 108 |
| FN | | | 12 | 13 | 11 | 16 | 13 | 12 |
| Sensitivity | | | 0.67 | 0.64 | 0.69 | 0.56 | 0.64 | 0.67 |
| Specificity | | | 0.72 | 0.74 | 0.72 | 0.76 | 0.72 | 0.79 |
| Success rate | | | 0.39 | 0.40 | 0.40 | 0.38 | 0.37 | 0.45 |

(8) Impact of the ΔΔG Threshold

In one embodiment, the threshold of 0 kcal/mol for predictors to select good mutations was used, i.e. mutations thought to improve the affinity to the antigen. As can be seen on Table 1, the SR for the double criterion (ΔΔEₚₒₗ*<threshold & ΔΔG*<threshold) does not vary much (from 39 to 45%) if the threshold varies between −1 and +1 kcal/mol. The main difference between different cutoffs is due to the total number of positives identified. The threshold could be used as a criterion to adjust the final number of proposed mutations to the capacities of the lab where the corresponding experiments are performed. In many embodiments of the present disclosure, the 0 kcal/mol threshold seems may be used by default.

(9) Predictor Success as Applied to Multiple Mutations in a Codon

In certain embodiments, the methods of the invention evaluate in silico affinity maturation of a library of mutations that are based on multiple codon changes. Accordingly, in the embodiments, the dataset excludes mutations with single codon base changes. For example, as shown in the Table 1, the additional exclusion of mutations with single codon base changes limited the exemplary dataset to 99 mutations, of which 22 were positives. The measured success rate on this focused dataset, when using the double criterion predictor of the invention (ΔΔEₚₒₗ*<0 kcal/mol & ΔΔG*<0 kcal/mol), is 63%. The ratio of positive mutations in the reduced set is identical to the one in the 159 mutations dataset, i.e. 22%.

In certain embodiments, the library of the mutations are based on at least two codon base changes since the most binding affinity enhancing mutations (ΔΔGₑₓₚ lower than −0.5 kcal/mol) are based on at least two codon base changes. For example, the method may be used to identify mutations with at least 2 codon base changes (ΔCodon-base>1), for which the binding is improved according to the double criteria predicator (ΔΔEₚₒₗ*<0 kcal/mol & ΔΔG*<0 kcal/mol).

In other embodiments, all the possible combinations of mutations (double, triple and possibly more) are evaluated. In one embodiment, a Generalized Born model is used instead of a Poisson Boltzmann model for increasing the speed in which the predictive calculations are made. In one embodiment, the explicit modelling of crystallographic waters is added as a step in the in silico protocol for identifying amino acid residues to mutate at the antibody-antigen interface because it is known that some of these water molecules mediate key interactions (Bhat, T. N et al., Bound water molecules and conformational stabilization help mediate an antigen-antibody association. Proc Natl Acad Sci USA 1994, 91, (3), 1089-93.

In other embodiments, the methods of the invention are extended by applying more CPU-intensive methods based for example on molecular dynamics simulations of the protein-protein interface. In particular, such simulations would quantify the entropic part of the binding event and thus refine the estimation of ΔG_{binding} (Zoete, V.; et al., MM-GBSA binding free energy decomposition and T cell receptor engineering. J Mol Recognit 2009, 23, (2), 142-52). In another embodiment, the additional step of explicit modelling of the backbone flexibility is added to the protocol depicted in FIG. 3.

(10) Computational Alanine Scanning.

In additional or alternative embodiments of the invention, alanine scanning analysis is performed on antibodies or other proteins to identify amino-acids which are crucial for binding. It is useful to optimize those amino acid residues that do not already contribute the most to successful binding of an antigen, so called “hot-spots”. When the crystallographic structure of the complex is available, the alanine scanning analysis can be performed by in silico alanine scanning techniques (Li, L. et al., Identification of hot spot residues at protein-protein interface. Bioinformation 2006, 1, (4), 121-6; Massova, et al., “Computational Alanine Scanning To Probe Protein-Protein Interactions: A Novel Approach To Evaluate Binding Free Energies. Journal of the American Chemical Society 1999, 121, (36), 8133-8143; Huo, S. et al., “Computational alanine scanning of the 1:1 human growth hormone-receptor complex”. J Comput Chem 2002, 23, (1), 15-27; Zoete, V.; Irving, M. B.; Michielin, O., MM-GBSA binding free energy decomposition and T cell receptor engineering. J Mol Recognit 2009, 23, (2), 142-52, incorporated herein by reference). In particular embodiments, in silico alanine scanning of a three dimensional high resolution antibody-antigen complex is use to focusing the randomization methods of the invention on key amino acid positions and is useful as a complementary approach to the design of specific variants containing a limited number of mutations.

### D. Synthesis of Focused Variant Antibody Library

By using the in silico based methods of the present disclosure, a focused library of variant proteins can be constructed and screened for candidates with desired functions (e.g., enhanced binding affinity). In some embodiments, a plurality of mutation, or variation, tolerant positions that improve (or at least do not negatively affect) the binding affinity of protein are introduced into the sequence of the parental antibody to generate a library of variants having alternate features. This library may comprise a subset of a ranked ordered list of variants. Nucleic acid molecules having predefined sequences that encode the variants may be synthesized to provide an expression library. These nucleic acid molecules may then be expressed to produce the binding protein variants which are screened to identify novel binding proteins or antibodies having the increased binding affinity.

In some embodiments, filtering techniques of the disclosure can be used to identify nucleic acid sequences to be included or excluded in a polypeptide expression library. For example, methods of the disclosure are useful for screening nucleic acid sequences that are candidates for inclusion in an expression library and identifying those sequences that encode polypeptides with one or more undesirable properties (e.g., poor solubility, high immunogenicity, low stability, etc.). Accordingly, aspects of the disclosure may be used to design a library of nucleic acids that encode a plurality of polypeptides having one or more biophysical or biological properties that are known or predicted to be within a predetermined acceptable or desirable range of values.

In certain embodiment the focused library may include more than about 100 different sequence variants (e.g., about 100, 1000, 2000. 3000, 4000, 5000, 6000, 7000, 8000, 9000, 10,000, 25,000, 50,000, 75,000, 100,000, 250,000, 500,000, 750,000, or 1,000,000 different sequences). In other embodiments, a relatively smaller expression library may be generated when unwanted polypeptide variants are excluded. For example, the number of clones required to represent all variants in a library will be smaller if the library is designed to exclude a subset of possible variants that are predicted to have unwanted traits. As a result, a relatively smaller library may be used to screen or select for a function or structure of interest when a subset of sequences is excluded from the library. Alternatively, a library of a predetermined size may be used to represent a higher number of potentially interesting polypeptide variants when unwanted variants are excluded. Accordingly, by excluding amino acid sequences that are predicted to have one or more unwanted traits, aspects of the disclosure may be useful to generate libraries that represent i) a higher number of potentially useful amino acid substitutions at a predetermined number of positions, or ii) potentially useful amino acid substitutions at more positions, or a combination thereof, relative to libraries that are not filtered.

### E. Analysis of Affinity

Affinity, avidity, and/or specificity can be measured in a variety of ways. Generally, and regardless of the precise manner in which affinity is defined or measured, the methods of the disclosure improve antibody affinity when they generate an antibody that is superior in any aspect of its clinical application to the antibody (or antibodies) from which it was made (for example, the methods of the disclosure are considered effective or successful when a modified antibody can be administered at a lower dose or less frequently or by a more convenient route of administration than an antibody (or antibodies) from which it was made).

More specifically, the affinity between an antibody and an antigen to which it binds can be measured by various assays, including, e.g., a BiaCore assay. Briefly, sepharose beads are coated with antigen such as a cancer antigen; a cell surface protein or secreted protein; an antigen of a pathogen (e.g., a bacterial or viral antigen (e.g., an HIV-antigen, an influenza antigen, or a hepatitis antigen)), or an allergen) by covalent attachment. (It is understood, however, that the methods described here are generally applicable; they are not limited to the production of antibodies that bind any particular antigen or class of antigens.

Those of ordinary skill in the art will recognize that determining affinity is not always as simple as looking at a single, bottom-line figure. Since antibodies have two arms, their apparent affinity is usually much higher than the intrinsic affinity between the variable region and the antigen, this phenomenon is also referred to as avidity. Intrinsic affinity can be measured using scFv or Fab fragments.

In certain embodiments, the relative affinities of the parent and modified antibodies (e.g., the parent, modified or altered antibody of the present disclosure) can be such that the affinity of the modified antibody to a given antigen is at least as high as the affinity of the parent antibody to that antigen. For example, the affinity of the modified antibody to the antigen can be at least (or about) 1.1, 1.2, 1.3, 1.4, 1.5, 1.6, 1.7, 1.8, 1.9, 2, 3, 5, 8, 10, 50, 10², 10³, 10⁴, 10⁵, or 10⁶, 10⁷, or 10⁸ times greater than the affinity of the parent antibody to the antigen (or any range or value in between).

The methods of the disclosure may be characterized as those that “produce” an antibody (or a fragment thereof). The term “produce” means to “make,” “generate,” or “design” a non-naturally occurring antibody (or fragment thereof). The antibody produced may be considered more “mature” than either of the antibodies whose sequences (e.g., whose CDR(s) and FRs) were used in its construction. While the antibody produced may have a stronger affinity for an antigen, the methods of the disclosure are not limited to those that produce antibodies with improved affinity. For example, the methods of the disclosure can produce an antibody that has about the same affinity for an antigen as it did prior to being modified by the present methods. When a human antibody is modified, as described in the prior art, to contain murine CDRs, the resulting CDR-grafted antibody can lose affinity for its antigen. Thus, for example, where the methods of the disclosure are applied to CDR-grafted antibodies, they are useful and successful when they prevent the loss of affinity (some or all of the loss) that would otherwise occur with a conventional CDR graft.

The method may also be used lower the affinity of the antibody, for example, where it is desirable to have a lower affinity for better pharmacokinetics, antigen-binding specificity, reduced cross-talk between related antigen epitopes, and the like. For example, the affinity of the modified antibody to the antigen can be at least (or about) 1.1, 1.2, 1.3, 1.4, 1.5, 1.6, 1.7, 1.8, 1.9, 2, 3, 5, 8, 10, 50, 10², 10³, 10⁴, 10⁵, or 10⁶, 10⁷, or 10⁸ times less than the affinity of the parent antibody to the antigen (or any range or value in between).

### F. Re-Screening

The methods of the disclosure can be iterative. An antibody identified and identified as having increased binding affinity, as described above, can be re-modeled (for example, in silico or empirically, e.g., using experimental data) and further altered to further improve antigen binding. Thus, the steps described above can be followed by additional steps, including: obtaining data corresponding to the structure of a complex between the modified antibody and the antigen; determining, using the data (which can be referred to as “additional data” to distinguish it from the data obtained and used in the parent “round”), a representation of an additional charge distribution of the CDRs of the modified antibody which minimizes electrostatic contribution to binding free energy between the modified antibody and the antigen; and expressing a third or further modified antibody that binds to the antigen, the third antibody having a matured CDR differing from a CDR of the modified antibody by at least one amino acid, the matured CDR corresponding to the additional charge distribution. Yet additional rounds of maturation can be carried out. In the method just described, the resulting antibody would be complexed with (i.e. allowed to bind to) antigen and used to obtain a free energy of binding. A fourth or further modified antibody would then be produced that would contain modifications, dictated by the identified point mutations, that improve antigen binding. And so forth.

### G. Construction of Modified Antibodies

Once the sequence of an antibody (e.g., a CDR-grafted or otherwise modified or “humanized” antibody) has been decided upon, that antibody can be made by techniques well known in the art of molecular biology. More specifically, recombinant DNA techniques can be used to produce a wide range of polypeptides by transforming a host cell with a nucleic acid sequence (e.g., a DNA sequence that encodes the desired protein products (e.g., a modified heavy or light chain; the variable domains thereof, or other antigen-binding fragments thereof)).

More specifically, the methods of production can be carried out as described above for chimeric antibodies. The DNA sequence encoding, for example, an altered variable domain can be prepared by oligonucleotide synthesis. The variable domain can be one that includes the FRs of a human acceptor molecule and the CDRs of a donor, e.g., murine, either before or after one or more of the residues (e.g., a residue within a CDR) has been modified to facilitate antigen binding. This is facilitated by determining the framework region sequence of the acceptor antibody and at least the CDR sequences of the donor antibody. Alternatively, the DNA sequence encoding the altered variable domain may be prepared by primer directed oligonucleotide site-directed mutagenesis. This technique involves hybridizing an oligonucleotide coding for a desired mutation with a single strand of DNA containing the mutation point and using the single strand as a template for extension of the oligonucleotide to produce a strand containing the mutation. This technique, in various forms, is described by, e.g., Zoller and Smith (Nuc. Acids Res. 10:6487-6500, 1982), Norris et al. (Nuc. Acids Res. 11:5103-5112, 1983), Zoller and Smith (DNA 3:479-488, 1984), and Kramer et al. (Nuc. Acids Res. 10:6475-6485, 1982).

Genetic codons may be used that can reduce the size chosen such that the diversity of the degenerate nucleic acid library of DNA segments within the experimentally coverable diversity. In one embodiment, genetic codons are used that require two or three nucleic acid base changes to effect a change in the amino acid residue for which they code for. Optionally, genetic codons that have more than one nucleic acid base change may be selected to code for amino acid substitutions for variant antibodies of the present disclosure.

Methods of the present disclosure may use genetic engineering techniques that may further comprise the steps of: building an amino acid positional variant profile of the CDR hit library; converting the amino acid positional variant profile of the CDR hit library into a first nucleic acid positional variant profile by back-translating the amino acid positional variants into their corresponding genetic codons; and constructing a degenerate CDR nucleic acid library of DNA segments by combinatorially combining the nucleic acid positional variants.

Other methods of introducing mutations into a sequence are known as well and can be used to generate the altered antibodies described herein (see, e.g., Carter et al., Nuc. Acids Res. 13:4431-4443, 1985). The oligonucleotides used for site-directed mutagenesis can be prepared by oligonucleotide synthesis or isolated from DNA coding for the variable domain of the donor antibody by use of suitable restriction enzymes.

### H. Host Cells and Cell Lines for Expression of the Modified Antibodies

Either the parent antibodies or modified antibodies as described herein (whether in a final form or an intermediate form) can be expressed by host cells or cell lines in culture. They can also be expressed in cells in vivo. The cell line that is transformed (e.g, transfected) to produce the altered antibody can be an immortalised mammalian cell line, such as those of lymphoid origin (e.g., a myeloma, hybridoma, trioma or quadroma cell line). The cell line can also include normal lymphoid cells, such as B-cells, that have been immortalized by transformation with a virus (e.g., the Epstein-Barr virus).

Although typically the cell line used to produce the altered antibody is a mammalian cell line, cell lines from other sources (such as bacteria and yeast) can also be used. In particular, E. coli-derived bacterial strains can be used, especially, e.g., phage display.

Some immortalized lymphoid cell lines, such as myeloma cell lines, in their normal state, secrete isolated Ig light or heavy chains. If such a cell line is transformed with a vector that expresses an altered antibody, prepared during the process of the disclosure, it will not be necessary to carry out the remaining steps of the process, provided that the normally secreted chain is complementary to the variable domain of the Ig chain encoded by the vector prepared earlier.

If the immortalized cell line does not secrete or does not secrete a complementary chain, it will be necessary to introduce into the cells a vector that encodes the appropriate complementary chain or fragment thereof.

In the case where the immortalized cell line secretes a complementary light or heavy chain, the transformed cell line may be produced for example by transforming a suitable bacterial cell with the vector and then fusing the bacterial cell with the immortalized cell line (e.g., by spheroplast fusion). Alternatively, the DNA may be directly introduced into the immortalized cell line by electroporation.

I. Pharmaceutical Formulations and their Uses

In prophylactic applications, pharmaceutical compositions or medicaments are administered to a subject suffering from a disorder in an amount sufficient to eliminate or reduce the risk, lessen the severity, or delay the outset of the disorder, including biochemical, histologic and/or behavioral symptoms of the disorder, its complications and intermediate pathological phenotypes presenting during development of the disorder. In therapeutic applications, compositions or medicaments are administered to a subject suspected of, or already suffering from such a disorder in an amount sufficient to cure, or at least partially arrest, the symptoms of the disorder (biochemical, histologic and/or behavioral), including its complications and intermediate pathological phenotypes in development of the disorder.

Effective doses of the compositions of the present disclosure, for the treatment of a condition vary depending upon many different factors, including means of administration, target site, physiological state of the subject, whether the subject is human or an animal, other medications administered, and whether treatment is prophylactic or therapeutic. Usually, the subject is a human but non-human mammals including transgenic mammals can also be treated.

For passive immunization with an antibody, the dosage ranges from about 0.0001 to 100 mg/kg, and more usually 0.01 to 20 mg/kg, of the host body weight. For example dosages can be 1 mg/kg body weight or 10 mg/kg body weight or within the range of 1-10 mg/kg, e.g., at least 1 mg/kg. Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered falls within the ranges indicated.

Antibodies are usually administered on multiple occasions. Intervals between single dosages can be weekly, monthly or yearly. In some methods, dosage is adjusted to achieve a plasma antibody concentration of 1-1000 mg/mL and in some methods 25-300 μg/mL. Alternatively, antibody can be administered as a sustained release formulation, in which case less frequent administration is required. Dosage and frequency vary depending on the half-life of the antibody in the subject. In general, human antibodies show the longest half-life, followed by humanized antibodies, chimeric antibodies, and nonhuman antibodies, in descending order.

The dosage and frequency of administration can vary depending on whether the treatment is prophylactic or therapeutic. In prophylactic applications, compositions containing the present antibodies or a cocktail thereof are administered to a subject not already in the disease state to enhance the subject's resistance. Such an amount is defined to be a “prophylactic effective dose.” In this use, the precise amounts again depend upon the subject's state of health and general immunity, but generally range from 0.1 to 25 mg per dose, especially 0.5 to 2.5 mg per dose. A relatively low dosage is administered at relatively infrequent intervals over a long period of time. Some subjects continue to receive treatment for the rest of their lives.

In therapeutic applications, a relatively high dosage (e.g., from about 1 to 200 mg of antibody per dose, with dosages of from 5 to 25 mg being more commonly used) at relatively short intervals is sometimes required until progression of the disease is reduced or terminated, and preferably until the subject shows partial or complete amelioration of symptoms of disease. Thereafter, the patient can be administered a prophylactic regime.

Therapeutic agents can be administered by parenteral, topical, intravenous, oral, subcutaneous, intraarterial, intracranial, intraperitoneal, intranasal or intramuscular means for prophylactic and/or therapeutic treatment. The most typical route of administration of a protein drug is intravascular, subcutaneous, or intramuscular, although other routes can be effective. In some methods, agents are injected directly into a particular tissue where deposits have accumulated, for example intracranial injection. In some methods, antibodies are administered as a sustained release composition or device. The protein drug can also be administered via the respiratory tract, e.g., using a dry powder inhalation device.

Agents of the disclosure can optionally be administered in combination with other agents that are at least partly effective in treatment of immune disorders.

The pharmaceutical compositions of the disclosure include at least one antibody of the disclosure in a pharmaceutically acceptable carrier. A “pharmaceutically acceptable carrier” refers to at least one component of a pharmaceutical preparation that is normally used for administration of active ingredients. As such, a carrier may contain any pharmaceutical excipient used in the art and any form of vehicle for administration. The compositions may be, for example, injectable solutions, aqueous suspensions or solutions, non-aqueous suspensions or solutions, solid and liquid oral formulations, salves, gels, ointments, intradermal patches, creams, lotions, tablets, capsules, sustained release formulations, and the like. Additional excipients may include, for example, colorants, taste-masking agents, solubility aids, suspension agents, compressing agents, enteric coatings, sustained release aids, and the like.

Agents of the disclosure are often administered as pharmaceutical compositions including an active therapeutic agent and a variety of other pharmaceutically acceptable components. See Remington's Pharmaceutical Science (15th ed., Mack Publishing Company, Easton, Pa. (1980)). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Antibodies can be administered in the form of a depot injection or implant preparation, which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl. Another example of a suitable formulation buffer for monoclonal antibodies contains 20 mM sodium citrate, pH 6.0, 10% sucrose, 0.1% Tween 80.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or microparticles such as polylactide, polyglycolide, or copolymer for enhanced adjuvant effect, as discussed above (see Langer, Science 249: 1527 (1990) and Hanes, Advanced Drug Delivery Reviews 28:97 (1997)).

### J. Therapies

Treatment of a subject suffering from a disease or disorder can be monitored using standard methods. Some methods entail determining a baseline value, for example, of an antibody level or profile in a subject, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically the individuals in the control population have not received prior treatment. Measured values of the level or profile in a subject after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

In other methods, a control value of the level or profile (e.g., a mean and standard deviation) is determined from a control population of individuals who have undergone treatment with a therapeutic agent and whose levels or profiles have plateaued in response to treatment. Measured values of levels or profiles in a subject are compared with the control value. If the measured level in a subject is not significantly different (e.g., more than one standard deviation) from the control value, treatment can be discontinued. If the level in a subject is significantly below the control value, continued administration of agent is warranted. If the level in the subject persists below the control value, then a change in treatment may be indicated.

In other methods, a subject who is not presently receiving treatment but has undergone a previous course of treatment is monitored for antibody levels or profiles to determine whether a resumption of treatment is required. The measured level or profile in the subject can be compared with a value previously achieved in the subject after a previous course of treatment. A significant decrease relative to the previous measurement (i.e., greater than a typical margin of error in repeat measurements of the same sample) is an indication that treatment can be resumed. Alternatively, the value measured in a subject can be compared with a control value (mean plus standard deviation) determined in a population of subjects after undergoing a course of treatment. Alternatively, the measured value in a subject can be compared with a control value in populations of prophylactically treated subjects who remain free of symptoms of disease, or populations of therapeutically treated subjects who show amelioration of disease characteristics. In all of these cases, a significant decrease relative to the control level (i.e., more than a standard deviation) is an indicator that treatment should be resumed in a subject.

The antibody profile following administration typically shows an immediate peak in antibody concentration followed by an exponential decay. Without a further dosage, the decay approaches pretreatment levels within a period of days to months depending on the half-life of the antibody administered. For example the half-life of some human antibodies is of the order of 20 days.

In some methods, a baseline measurement of antibody to a given antigen in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dosage of antibody is administered. In some methods, peak or subsequent measured levels less background are compared with reference levels previously determined to constitute a beneficial prophylactic or therapeutic treatment regime in other subjects. If the measured antibody level is significantly less than a reference level (e.g., less than the mean minus one standard deviation of the reference value in population of subjects benefiting from treatment) administration of an additional dosage of antibody is indicated.

### EXAMPLES

The following examples are included for purposes of illustration and should not be construed as limiting the disclosure.

### Example 1

### In Silico Affinity Optimization of Antibodies and Enzymes Using a Double Criterion Predictor

An in silico protocol was applied to 7 protein systems: an anti-VLA1 antibody, the anti-lysozymes D44.1, D1.3 and HyHEL-63 antibodies, the anti-EGFR antibody (Cetuximab/Erbitux/IMC-C225), the anti-HER2 (Herceptin) variant bH1, and the Barnase-Barstar complex. Experimentally, the effect of a mutation can be considered as positive when the measured impact on binding is favourable (ΔΔG_{binding}<0 kcal/mol). Conversely, the effect of a mutation can be considered as negative when the measured impact on binding is unfavourable (ΔΔG_{binding}>0 kcal/mol). Two criteria were applied for the classification of mutations using computed values: a mutation was predicted positive if both the calculated impact on binding (ΔΔG_{binding}<cutoff), and antibody stability, were favourable. The initial cutoff used to predict positive mutations was 0 kcal/mol. Mutations for which no conformation was found by DEE/A* sampling or for which the sampling algorithm did not converge were considered as negative.

The results for the 6 variations of the computational protocol are summarized in Table1. For this evaluation, 173 mutations of 7 protein complexes with available experimental data, have been used. The 6 variations differ in the way the free energy of binding was calculated, which is subsequently used to predict if a mutation will have a beneficial or detrimental impact on binding. Thus, the six energies were referred to as “predictors” as detailed above and in the tables below.

For the D44.1, D1.3 and Cetuximab systems, 36 mutations are reported, of which 14 were shown experimentally to improve the binding between the antibody and the antigen (positive mutations). Using ΔΔG* as a predictor (calculated ΔΔG_{binding}, averaged as described in Equation 4), 10 true positives (TP) and 6 false positives (FP) were found. The sensitivity and specificity values for this predictor were 0.71 and 0.73, respectively. The best performances were obtained by the top ΔΔEₚₒₗ predictor, with a success rate of 73% . The success rates (SR) for other predictors are: SR_{AAG*}=63%, SR_{ΔΔG**}=60%, SR_{ΔΔEpol*}=69%, SR_{ΔΔGtop}=64% and SR_{ΔΔG* & ΔΔEpol*}=71%.

For the Anti-VLA1 system, 67 mutations were available⁹, of which 10 were found to be positive. 70% of all positive mutations were found with the ΔΔG* predictor (sensitivity=0.7), and the specificity reached 0.67. The success rate of the double criteria predictor (ΔΔG* & ΔΔEₚₒₗ) was improved when compared to the ΔΔG* predictor (SR_{ΔΔG* & ΔΔEpol*}=35% and SR_{ΔΔG*}=27% respectively). This is due to a significantly lower number of false positives (13 and 19, respectively).

For HHEL-63 and HER2 complexes, only mutations into Ala were available (alanine scanning) The ΔΔG* predictor identifies 7 of the 11 positive mutations, i.e. hot spots of interaction, and 36 out of the 45 negatives (Sensitivity=0.64 and specificity=0.8).

The Barnase-Barstar is a system that is well known for its optimized interface. As a consequence, the mutations found in the literature are mainly unfavorable (1 positive and 13 negative mutations). The ΔΔG* predictor has a specificity of 0.69, and the positive mutation was predicted by none of the predictors.

The global performance on all 7 systems is as follows (“All” part of the Table 1 above): The sensitivity varies in the [0.56-0.69] range, while the specificity varies in the [0.72-0.79] range. More importantly, the success rate (% of positive mutations in the selected subset) varies between 37% and 45%. The best performances were obtained with the double criteria predictor (ΔΔG*<0 kcal/mol & ΔΔEₚₒₗ*<0 kcal/mol) with a sensitivity of 0.67 and a success rate of 45%.

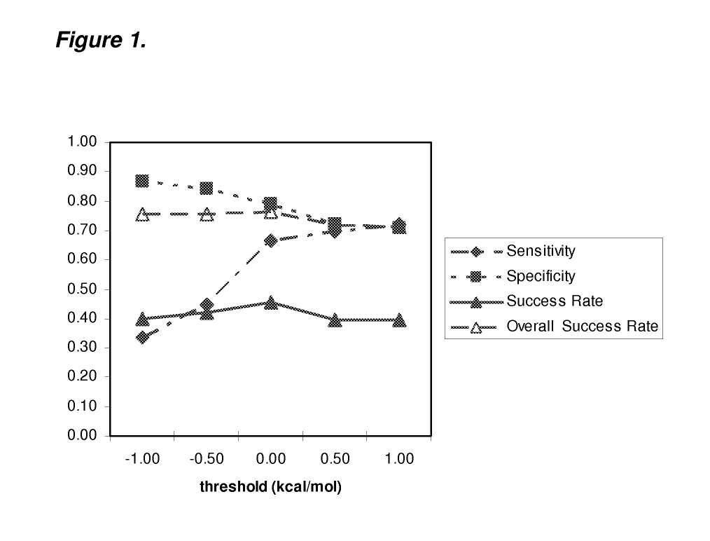

The impact of the predicted ΔΔG_{binding} threshold on predictive performances was compared using different thresholds governing the decision that a mutation is predicted positive. Several cutoff values (−1, −0.5, 0, 0.5 and 1 kcal/mol) were tested for the ΔΔG* and ΔΔEₚₒₗ* predictors (Table 2). As expected, a lower cutoff led to a lower sensitivity (ie less positives detected), and a higher specificity (i.e more negatives found). The effect of the cutoff on the performances of the double criterion (ΔΔG* & ΔΔEₚₒₗ*) is illustrated in FIG. 1. Interestingly, the success rates (SR) remain stable for each predictor across the different cutoff values.

Table 2 depicts an evaluation of ΔΔG* and ΔΔEₚₒₗ predictors at different cutoff values. The whole data set was taken into account. Cutoff refers to the threshold used to select positive mutations by an in silico predictor. The cutoff values listed apply to the results shown in the respective cells. Definitions of ΔΔG* and ΔΔEₚₒₗ* are described in the caption of Table 2. Numbers that are not in bold refer to the number of TP, FP, TN or FN.

TABLE 2

| | | Exp. | | | |
|---|---|---|---|---|---|
| | Cutoff | (threshold = | | | ΔΔG* & |
| | (kcal/mol) | 0 kcal/mol) | ΔΔG* | ΔΔEpol* | ΔΔEpol* |
| TP | −1 | 36 | 15 | 15 | 12 |
| FP | | | 21 | 25 | 18 |
| TN | | 137 | 116 | 112 | 119 |
| FN | | | 21 | 21 | 24 |
| Sensitivity | | | 0.42 | 0.42 | 0.33 |
| Specificity | | | 0.85 | 0.82 | 0.87 |
| Success rate | | | 0.42 | 0.38 | 0.40 |
| TP | −0.5 | 36 | 17 | 20 | 16 |
| FP | | | 29 | 29 | 22 |
| TN | | 137 | 108 | 108 | 115 |
| FN | | | 19 | 16 | 20 |
| Sensitivity | | | 0.47 | 0.56 | 0.44 |
| Specificity | | | 0.79 | 0.79 | 0.84 |
| Success rate | | | 0.37 | 0.41 | 0.42 |
| TP | 0 | 36 | 24 | 25 | 24 |
| FP | | | 38 | 38 | 29 |
| TN | | 137 | 99 | 99 | 108 |
| FN | | | 12 | 11 | 12 |
| Sensitivity | | | 0.67 | 0.69 | 0.67 |
| Specificity | | | 0.72 | 0.72 | 0.79 |
| Success rate | | | 0.39 | 0.40 | 0.45 |
| TP | 0.5 | 36 | 26 | 26 | 25 |
| FP | | | 43 | 47 | 38 |
| TN | | 137 | 94 | 90 | 99 |
| FN | | | 10 | 10 | 11 |
| Sensitivity | | | 0.72 | 0.72 | 0.69 |
| Specificity | | | 0.69 | 0.66 | 0.72 |
| Success rate | | | 0.38 | 0.36 | 0.40 |
| TP | 1 | 36 | 26 | 29 | 26 |
| FP | | | 44 | 54 | 40 |
| TN | | 137 | 93 | 83 | 97 |
| FN | | | 10 | 7 | 10 |
| Sensitivity | | | 0.72 | 0.81 | 0.72 |
| Specificity | | | 0.68 | 0.61 | 0.71 |
| Success rate | | | 0.37 | 0.35 | 0.39 |

### Example 2

### In Silico Affinity Optimization with a Focused Data Set Having Multiple Base Changes Per Codon

The performance of the predictors on the mutations generated is less likely to have been sampled by directed evolution or experimental methods. A more focused data set was compiled by selecting only those mutations with the property Δcodon-base above 1. Δcodon-base is the minimal number of base changes at the DNA level required to generate a given mutation from the wild-type. Also, in order to focus the analysis on antibody engineering applications, the Barnase-Barstar system was not taken into account. As shown in Table 3, the predictive performances were significantly better on this subset. 63% of predicted positives (using the double criterion ΔΔG*<0 & ΔΔEₚₒₗ*<0) are indeed affinity enhancing mutations, as compared to 49% for the whole dataset.

Table 3 depicts a comparison of three predictors for the whole data set or the “ΔCodon-base>1” focused data set. The Barnase-Barstar complex was not taken into account. ΔΔG* and ΔΔEₚₒₗ* are defined as outlined above. The criteria to predict positive mutations on the remaining dataset were ΔΔG*<0, ΔΔEₚₒₗ*<0, or (ΔΔG*<0 & ΔΔEₚₒₗ*<0).

TABLE 1

| | | | | | ΔΔG* & |
|---|---|---|---|---|---|
| | Dataset | Exp. | ΔΔG* | ΔΔEpol* | ΔΔEpol* |
| TP | Large | 35 | 24 | 25 | 24 |
| FP | | | 34 | 33 | 25 |
| TN | | 124 | 90 | 91 | 99 |
| FN | | | 11 | 10 | 11 |
| Sensitivity | | | 0.69 | 0.71 | 0.69 |
| Specificity | | | 0.73 | 0.73 | 0.80 |
| Success rate | | | 0.41 | 0.43 | 0.49 |
| TP | Focused | 22 | 15 | 15 | 15 |
| FP | | | 16 | 14 | 9 |
| TN | | 77 | 61 | 63 | 68 |
| FN | | | 7 | 7 | 7 |
| Sensitivity | | | 0.68 | 0.68 | 0.68 |
| Specificity | | | 0.79 | 0.82 | 0.88 |
| Sensitivity^{o} | | | 0.43 | 0.43 | 0.43 |
| Specificity^{c} | | | 0.49 | 0.51 | 0.55 |
| Success rate | | | 0.48 | 0.52 | 0.63 |

### Example 3

### Computational Alanine Scanning

To evaluate the performances of the predictors in predicting hot-spots by computational alanine scanning, the dataset was reduced to the two systems (anti-HER2 and HyHEL-63) for which only alanine mutations were available, see Table 4 which depicts results for computational alanine scanning applied to HER2 and HyHEL-63. ΔΔG* and ΔΔEₚₒₗ*.

To be consistent with the principle of hot-spot detection, where the hot-spot is defined as an amino acid with a critical contribution to the overall binding, negative mutations were defined as mutations with an experimental ΔΔG_{binding} of at least 1 kcal/mol. The sensitivity and specificity were calculated as usual, but the success rate does not apply anymore to the positives, and was calculated as follows: SR=TN/(TN+FN).

In Table 5, a comparison of the hot-spots predicted in silico versus the experimental hot-spots is shown. Table 5 depicts residues of HER2 identified as hot-spots by the in vitro alanine scanning (in black if ΔΔG_{binding}>1 kcal/mol, in grey if ΔΔG_{binding}>0.5 kcal/mol), or by in silico alanine scanning (in black if ΔΔG_{binding}>1 kcal/mol). The remaining residues (white cells) are considered optimizable.

Table 6 depicts mutations in D44.1, Cetuximab, and D1.3 systems, with the corresponding experimental and in silico data. ΔΔG* and ΔΔEₚₒₗ* are the ΔΔG_{binding} and ΔΔEₚₒₗ averaged by Equation 4, and ΔΔG** is the ΔΔG_{binding} averaged by Equation 3. The top ΔΔG and top ΔΔEₚₒₗ are the lowest ΔΔG or ΔΔEₚₒₗ among sampled conformations. A mutation is considered as not unstable by EGAD if the stability energy is lower than 3 kcal/mol, or if the clash score is between −2 and +2. The ΔCodon-base is the minimum of DNAs change to generate the mutation.

Table 7 depicts mutations in antiVLA1 antibody-antigen complex with the corresponding experimental and in silico data.

Table 8 depicts mutations in Barnase-Barstar complex, and in the HyHEL-63 antibody-antigen complex, with the corresponding experimental and in silico data.

Table 9 depicts mutations in bH1-Herceptin antibody-antigen complex, with the corresponding experimental and in silico data.

TABLE 2
Computational Alanine Scanning of HER2 and HyHEL-63

| | | Exp. | | | |
|---|---|---|---|---|---|
| | Cutoff | (threshold = | | | ΔΔG* & |
| | (kcal/mol) | 1 kcal/mol) | ΔΔG* | ΔΔEpol* | ΔΔEpol* |
| TP | −1 | 28 | 2 | 4 | 2 |
| FP | | | 2 | 3 | 2 |
| TN | | 28 | 26 | 25 | 26 |
| FN | | | 26 | 24 | 26 |
| Sensitivity | | | 0.07 | 0.14 | 0.07 |
| Specificity | | | 0.93 | 0.89 | 0.93 |
| Success rate | | | 0.50 | 0.51 | 0.50 |
| TP | −0.5 | 28 | 6 | 7 | 4 |
| FP | | | 2 | 3 | 2 |
| TN | | 28 | 26 | 25 | 26 |
| FN | | | 22 | 21 | 24 |
| Sensitivity | | | 0.21 | 0.25 | 0.14 |
| Specificity | | | 0.93 | 0.89 | 0.93 |
| Success rate | | | 0.54 | 0.54 | 0.52 |
| TP | 0 | 28 | 13 | 15 | 12 |
| FP | | | 3 | 5 | 3 |
| TN | | 28 | 25 | 23 | 15 |
| FN | | | 13 | 13 | 16 |
| Sensitivity | | | 0.50 | 0.54 | 0.43 |
| Specificity | | | 0.89 | 0.82 | 0.83 |
| Success rate | | | 0.66 | 0.64 | 0.48 |
| TP | 0.5 | 28 | 16 | 19 | 16 |
| FP | | | 3 | 6 | 3 |
| TN | | 28 | 25 | 22 | 25 |
| FN | | | 12 | 9 | 12 |
| Sensitivity | | | 0.57 | 0.68 | 0.57 |
| Specificity | | | 0.89 | 0.79 | 0.89 |
| Success rate | | | 0.68 | 0.71 | 0.68 |
| TP | 1 | 28 | 17 | 22 | 17 |
| FP | | | 3 | 6 | 3 |
| TN | | 28 | 25 | 22 | 15 |
| FN | | | 11 | 6 | 11 |
| Sensitivity | | | 0.61 | 0.79 | 0.61 |
| Specificity | | | 0.89 | 0.79 | 0.83 |
| Success rate | | | 0.69 | 0.79 | 0.58 |

TABLE 3
Identition of Hot spots in HER2.

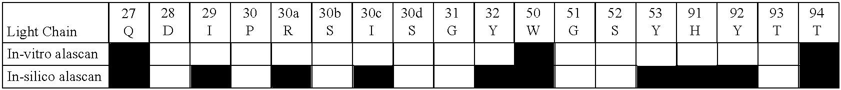

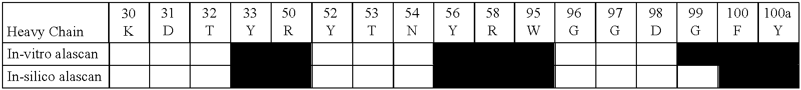

TABLE 4
Mutations in D44.1, Cetuximab and D.13 Antibodies

| | | | | | | EGAD | EGAD | | | top | top |
|---|---|---|---|---|---|---|---|---|---|---|---|
| System | Position | Mutation | ΔΔGexp | ΔΔG* | ΔΔG** | stability | clashes | ΔΔEpol* | ΔCodon | ΔΔG | ΔΔEpol |
| D44.1 | L32 Asn | Gly | −0.86 | −1.98 | −1.99 | — | — | −3.82 | 2 | 0.58 | −1.65 |
| D44.1 | L32 Asn | Tyr | 0 | −1.2 | −1.15 | 1.04 | 0 | −6.01 | 1 | 0.57 | 1.13 |
| D44.1 | L92 Asn | Ala | −1.25 | −1.16 | −0.81 | 1.1 | 0 | −3.88 | 2 | 1.08 | −0.79 |
| D44.1 | H28 Thr | Asp | −0.15 | −0.08 | 1.54 | −2.14 | 0 | −2.14 | 2 | −0.01 | 0.6 |
| D44.1 | H31 Thr | Ala | 0.45 | 1.55 | −2.59 | −1.84 | 0 | −0.51 | 1 | 1.87 | −0.18 |
| D44.1 | H31 Thr | Val | 0.53 | −2.35 | −2.59 | −2.25 | 1 | −1.74 | 2 | −2.46 | −0.18 |
| D44.1 | H31 Thr | Trp | 0.13 | −2.81 | −2.81 | −2.82 | 1 | −1.39 | 2 | −2.66 | −1.17 |
| D44.1 | H57 Ser | Ala | −0.37 | −3.76 | −3.59 | −0.17 | 0 | −4.26 | 1 | −1.95 | −3.19 |
| D44.1 | H57 Ser | Val | −0.49 | −4.14 | −4.82 | 2.74 | 5 | −4.23 | 2 | −3.46 | −2.8 |
| D44.1 | H58 Thr | Asp | −0.56 | −4.49 | −4.52 | 0.33 | −1 | −4.64 | 2 | −4.49 | −4.62 |
| D44.1 | H65 Lys | Asp | 0.02 | −4.73 | −4.72 | 1.32 | 0 | −4.51 | 2 | −4.74 | −4.53 |
| Cetuximab | L26 Ser | Asp | −0.2 | −1.90 | −1.88 | −0.16 | 0 | −2.00 | 2 | −1.84 | −1.95 |
| Cetuximab | L31 Thr | Glu | −0.5 | −1.66 | −1.76 | −0.42 | 0 | −2.17 | 2 | −1.78 | −1.86 |
| Cetuximab | L93 Asn | Ala | −0.7 | 2.65 | 1.84 | 0.64 | 0 | 0.83 | 2 | 0.06 | −1.07 |
| Cetuximab | H56 Asn | Ala | −0.04 | −0.10 | −0.03 | 0.63 | −1 | −2.17 | 2 | 0 | −2.05 |
| Cetuximab | H61 Thr | Glu | −0.07 | −2.18 | −2.16 | −3.16 | 1 | −2.25 | 2 | −2.55 | −2.74 |
| D1.3 | H28 Ser | Asn | 0.15 | 1.45 | 1.48 | −0.57 | 0 | 1.28 | 1 | 1.08 | 1.01 |
| D1.3 | H28 Ser | Gln | 0.08 | 1.57 | 0.56 | 0.04 | 0 | 0.61 | 2 | 0.41 | 0.26 |
| D1.3 | H28 Ser | Asp | 0 | 0.03 | 0.16 | −0.8 | 0 | 0.33 | 2 | 0.14 | 0.33 |
| D1.3 | H28 Ser | Glu | −0.11 | −1.59 | −1.55 | 0.94 | 0 | −0.77 | 2 | −1.56 | −0.78 |
| D1.3 | H31 Gly | Ala | 0.3 | 1.39 | 1.37 | — | — | 1.47 | 1 | 1 | 1.08 |
| D1.3 | H31 Gly | Glu | −0.5 | 4.43 | 4.35 | — | — | −5.14 | 1 | 4.43 | −5.06 |
| D1.3 | H31 Gly | Trp | 0 | 5.3 | 5.17 | — | — | 6.66 | 1 | 4.67 | 6.21 |
| D1.3 | H32 Tyr | Glu | 1.9 | −1.63 | −1.63 | 4.41 | 1 | −3.69 | 2 | −2.68 | −8.02 |
| D1.3 | H58 Asp | Asn | −0.1 | 1.47 | 1.61 | 0.70 | 0 | 2.10 | 1 | 1.65 | 2.08 |
| D1.3 | H58 Asp | Glu | 0.1 | 1.37 | 1.07 | 0.60 | 0 | 0.61 | 1 | 0.65 | 0.71 |
| D1.3 | H99 Arg | Tyr | 1.2 | 13.65 | 13.68 | 2.30 | 4 | 4.15 | 2 | 13.65 | 4.13 |
| D1.3 | H99 Arg | Trp | 0.7 | 15.99 | 11.81 | 1.28 | 1 | 2.42 | 1 | 0.91 | 1.92 |
| D1.3 | L31 Asn | Trp | 0.2 | −0.51 | 0.79 | 0.65 | 0 | 2.64 | 3 | 0.74 | 2.59 |
| D1.3 | L32 Tyr | Trp | >+5 | np | np | 2.05 | 4 | np | 2 | np | np |
| D1.3 | L46 Leu | Asp | >+5 | −0.13 | −0.14 | 1.95 | −1 | 0.22 | 2 | −0.19 | 0.18 |
| D1.3 | L46 Leu | Glu | >+5 | −0.55 | −0.55 | 4.01 | 1 | −0.07 | 2 | −0.66 | −0.18 |
| D1.3 | L50 Tyr | Arg | 0.85 | 5.46 | 4.81 | 3.84 | 1 | 1.23 | 2 | 4.91 | 3.54 |
| D1.3 | L50 Tyr | Lys | 1.71 | 10.32 | 10.27 | 3.37 | 1 | −0.37 | 2 | 10.73 | 1.69 |
| D1.3 | L52 Thr | Phe | 0.48 | 0.34 | 0.5 | −1.51 | 0 | 0.39 | 2 | 0.43 | 2.26 |
| D1.3 | L53 Thr | Arg | 1.6 | 13.07 | 12.96 | −0.41 | 1 | 11.09 | 1 | 12.83 | 10.74 |

TABLE 7
Mutations in anti-VLA antibody-antigen complex

| | | | | | | EGAD | Egad | | | top | top |
|---|---|---|---|---|---|---|---|---|---|---|---|
| System | Position | Mutation | ΔΔGexp | ΔΔG* | ΔΔG** | stability | clashes | ΔΔEpol* | ΔCodon | ΔΔG | ΔΔEpol |
| antiVLA1 | L30 Asn | Val | 1.1 | 2.58 | −5.29 | 6.84 | 4 | −5.02 | 2 | −5.62 | −5.31 |
| antiVLA1 | L31 His | Arg | 1.1 | −6.47 | −4.74 | 2.6 | 2 | −23.76 | 1 | −4.74 | −23.84 |
| antiVLA1 | L31 His | Lys | 1.2 | −7.41 | −5.9 | 3.52 | 3 | −14.97 | 2 | −6.38 | −14.77 |
| antiVLA1 | L49 Leu | Lys | 0.8 | −0.49 | 0.77 | 0.42 | 0 | −3.43 | 2 | 0.45 | −3.49 |
| antiVLA1 | L52 Asn | Arg | 0.9 | 10.19 | 11.67 | 0.3 | 0 | 16.57 | 2 | 11.34 | 16.47 |
| antiVLA1 | L52 Asn | Lys | 0.7 | 10.03 | 11.85 | 0.56 | 0 | 13.55 | 1 | 11.91 | 13.61 |
| antiVLA1 | H31 Arg | Gln | 1.3 | −9.52 | −9.44 | 0.76 | 0 | −12.29 | 1 | −9.56 | −12.21 |
| antiVLA1 | H35 Ser | Gln | >1 | −2.32 | −2.2 | 22.04 | 11 | −2.42 | 2 | −0.91 | −1.01 |
| antiVLA1 | H50 Thr | Val | −0.3 | −2.22 | −2.11 | 0.19 | 1 | −2.07 | 2 | −0.89 | −0.87 |
| antiVLA1 | H59 Tyr | Glu | 0.5 | −0.04 | 0.3 | 1.61 | 0 | 0.23 | 2 | −1.02 | −1.01 |
| antiVLA1 | H60 Leu | Asp | −0.1 | −1.42 | −1.38 | 2.98 | 0 | −1.38 | 2 | −1.34 | −1.35 |
| antiVLA1 | H64Lys | Glu | −0.2 | −1.58 | −1.28 | 0.58 | 0 | −1.34 | 1 | −0.89 | −0.98 |
| antiVLA1 | H64Lys | Gln | −0.2 | −1.19 | −0.86 | 0.69 | 0 | −0.78 | 1 | −0.37 | −0.39 |
| antiVLA1 | H64Lys | Asp | −0.1 | −1.73 | −1.4 | 1.77 | 0 | −1.33 | 2 | −0.93 | −0.93 |
| antiVLA1 | H64Lys | Asn | −0.1 | −1.27 | −0.92 | 1.05 | 0 | −0.86 | 1 | −0.39 | −0.4 |
| antiVLA1 | L28 Ser | Gln | −0.7 | 0.2 | 0.24 | −0.27 | −1 | 0.91 | 2 | −0.85 | 1.66 |
| antiVLA1 | L30 Asn | Tyr | >1 | 5.15 | 5.06 | 25.09 | 13 | −0.38 | 1 | 3.48 | −2.52 |
| antiVLA1 | L30 Asn | Trp | >1 | 11.93 | 7.9 | 27.18 | 18 | 6.6 | 3 | 7.36 | 6.05 |
| antiVLA1 | L52 Asn | Tyr | −0.1 | 1.69 | 0.87 | −0.52 | 1 | 3.28 | 1 | 0.52 | 2.85 |
| antiVLA1 | L91 Ser | Thr | >1 | 4.83 | 1.38 | 1.91 | 0 | −0.12 | 1 | 1.19 | −0.43 |
| antiVLA1 | L91 Ser | Arg | >1 | −2 | −1.98 | 38.89 | 18 | 8.78 | 1 | 3.24 | 6.98 |
| antiVLA1 | L92 Gly | Gln | >1 | −0.03 | −0.01 | — | — | −0.33 | 2 | −0.02 | −0.33 |
| antiVLA1 | L92 Gly | Ser | 0.5 | −0.84 | −0.83 | — | — | −0.71 | 1 | −0.82 | −0.71 |
| antiVLA1 | H52 Ser | Met | >1 | −13.14 | −12.5 | 34.98 | 23 | −11.02 | 2 | −5.84 | −3.83 |
| antiVLA1 | H53 Gly | Ser | 0.7 | −0.99 | −3.13 | — | — | −3.11 | 1 | −3.91 | −3.02 |
| antiVLA1 | H53 Gly | Ala | 0.5 | −2.3 | −2.31 | — | — | −1.27 | 1 | −2.36 | −1.3 |
| antiVLA1 | H54 Gly | Ile | 1.3 | −0.68 | −0.57 | — | — | 0.16 | 2 | −0.55 | 0.36 |
| antiVLA1 | H54 Gly | Thr | 1.2 | −1.43 | −1.34 | — | — | 0.6 | 2 | −1.38 | 0.75 |
| antiVLA1 | H54 Gly | Met | NB | −1.5 | −1.76 | — | — | 1.45 | 2 | −2.1 | 3.3 |
| antiVLA1 | H58 Tyr | Gln | >1 | 7.08 | 3.89 | 5.51 | 1 | −4.87 | 2 | 3.97 | −4.87 |
| antiVLA1 | H58 Tyr | Glu | >1 | 24.28 | 23.92 | 4.57 | 1 | 12.54 | 2 | 23.97 | 12.55 |
| antiVLA1 | H58 Tyr | Trp | 0.8 | 6.8 | 4.29 | 0.05 | 0 | −0.87 | 2 | 1.04 | −1.7 |
| antiVLA1 | H100 Gly | Met | 0.1 | 42.19 | 35.23 | — | — | 42.13 | 2 | 35.05 | 42.69 |
| antiVLA1 | H100 Gly | Val | NB | 47.25 | 47.58 | — | — | 46.42 | 1 | 47.84 | 46.67 |
| antiVLA1 | H100 Gly | Ile | NB | 50.22 | 50.46 | — | — | 50.11 | 2 | 50.71 | 50.3 |
| antiVLA1 | L24 Ser | Arg | −0.1 | −0.37 | −0.41 | −0.65 | 0 | −0.29 | 1 | −0.44 | −0.36 |
| antiVLA1 | L49 Leu | Tyr | NB | −8.31 | −6.53 | 1.31 | 2 | −9.63 | 2 | −6.58 | −9.42 |
| antiVLA1 | L49 Leu | Phe | 0.7 | −7.05 | −6.52 | 0.07 | 0 | −5.65 | 1 | −6.66 | −5.8 |
| antiVLA1 | L49 Leu | Trp | >1 | np | np | 5.9 | 7 | np | 1 | np | np |
| antiVLA1 | L52 Asn | Asp | 0.5 | 1.76 | 0.63 | 0.31 | 0 | 0.66 | 1 | 0.56 | 0.63 |
| antiVLA1 | L52 Asn | Glu | −0.2 | 1.43 | 1.12 | 0.84 | 0 | 0.54 | 2 | 0.96 | 0.7 |
| antiVLA1 | L90 Trp | Gln | >1 | −2.88 | −2.68 | −42.95 | −28 | −5.34 | 2 | −3.41 | −15.92 |
| antiVLA1 | L91 Ser | Gln | >1 | 2.3 | 2.24 | 3.01 | 2 | 8.2 | 2 | 6.13 | 5.7 |
| antiVLA1 | L91 Ser | Trp | NB | −8.77 | −8.75 | 2.59 | 5 | 0.09 | 1 | −3.51 | −1.73 |
| antiVLA1 | H33 Thr | Val | 0.4 | 2.97 | −2.55 | 2.76 | 4 | −0.69 | 2 | −2.51 | −0.61 |
| antiVLA1 | H33 Thr | Asn | >1 | −0.76 | −0.52 | 1.04 | −2 | 1.02 | 1 | −0.57 | 1.53 |
| antiVLA1 | H33 Thr | Gln | NB | −0.78 | −0.66 | −2.04 | −1 | −2.51 | 2 | −1.06 | −2.92 |
| antiVLA1 | H35 Ser | Ala | >1 | −0.47 | −0.42 | −1.2 | 0 | −0.41 | 1 | 0.27 | −0.17 |
| antiVLA1 | H35 Ser | Val | NB | −3.49 | −3.11 | 8.83 | 4 | −3 | 2 | −2.03 | −2.3 |
| antiVLA1 | H50 Thr | Gln | NB | 4.61 | 4.67 | 5.53 | 5 | 4.96 | 2 | −2.55 | −1.54 |
| antiVLA1 | H50 Thr | Glu | NB | 31.81 | 31.9 | 2.74 | 4 | 31.8 | 2 | 28.84 | 29.75 |
| antiVLA1 | H52 Ser | Thr | NB | −3.82 | −7.29 | 11.24 | 7 | −7.51 | 1 | 0.97 | −0.8 |
| antiVLA1 | H53 Gly | Trp | NB | np | np | — | — | np | 1 | np | np |
| antiVLA1 | H53 Gly | Gln | NB | 18.52 | 17.71 | — | — | 2.8 | 2 | 17.06 | 2.88 |
| antiVLA1 | H54 Gly | Asn | NB | −1.29 | −10.17 | — | — | −6.69 | 2 | −10.28 | −6.84 |
| antiVLA1 | H56 His | Tyr | NB | −1.03 | −1.61 | 15.33 | 8 | 0.04 | 1 | −1.25 | 0.56 |
| antiVLA1 | H99 Phe | Trp | 0 | 1.42 | 1.19 | -0.67 | 0 | 2.75 | 2 | 0.93 | 2.61 |
| antiVLA1 | H99 Phe | Tyr | 0.8 | 2.5 | 2.47 | 0.11 | 0 | 2.68 | 1 | 1.74 | 2.19 |
| antiVLA1 | H100 Gly | Leu | >1 | 48.22 | 28.23 | — | — | 33.21 | 2 | 39.7 | 46.71 |
| antiVLA1 | H100 Gly | Phe | 0.6 | np | np | — | — | np | 2 | np | np |
| antiVLA1 | H100 Gly | Ser | 0.6 | 23.54 | 22.57 | — | — | 18.34 | 1 | 26.7 | 19.48 |
| antiVLA1 | H102 Gly | Ser | NB | −5.84 | −5.67 | — | — | −2.75 | 1 | −5.68 | −2.77 |
| antiVLA1 | L31 His | Trp | 1.3 | 2.73 | 1.67 | 5.72 | 4 | 5.03 | 3 | 1.55 | 4.88 |
| antiVLA1 | H53 Gly | Asn | 1.3 | 13.83 | 9.81 | — | — | 3.29 | 2 | −5.66 | −7.81 |
| antiVLA1 | H54 Gly | Tyr | 0 | −5.79 | −5.61 | — | — | 1.93 | 2 | −5.8 | 1.73 |
| antiVLA1 | H99 Phe | Trp | 0 | 1.42 | 1.19 | −0.67 | 0 | 2.75 | 2 | 0.93 | 2.61 |
| antiVLA1 | H99 Phe | Tyr | 0.8 | 2.5 | 2.47 | 0.11 | 0 | 2.68 | 1 | 1.74 | 2.19 |

TABLE 8
Mutations in Barnase-Barstar Complex

| | | | | | | EGAD | Egad | | | top | top |
|---|---|---|---|---|---|---|---|---|---|---|---|
| System | Position | Mutation | ΔΔGexp | ΔΔG* | ΔΔG** | stability | clashes | ΔΔEpol* | ΔCodon | ΔΔG | ΔΔEpol |
| BnBs | Bn27 Lys | Ala | 5.4 | −4.46 | −3.2 | 2.99 | −1 | −8.89 | 2 | −3.36 | −9 |
| BnBs | Bn59 Arg | Ala | 5.2 | −2.31 | −2.48 | 3.5 | 0 | −4.81 | 2 | −2.12 | −2.51 |
| BnBs | Bn83 Arg | Gln | 5.4 | −3.7 | −7.84 | 2.05 | 1 | −9.62 | 1 | −5.58 | −6.8 |
| BnBs | Bn87 Arg | Ala | 5.5 | −15.86 | −16.58 | −25.12 | −12 | −14.43 | 2 | −20.91 | −27.06 |
| BnBs | Bn102 His | Ala | 6.1 | 13.37 | 13.3 | 1.77 | −1 | 5.53 | 2 | 13.38 | 6.57 |
| BnBs | Bs29 Tyr | Phe | −0.1 | 2.64 | 2.81 | −0.27 | 0 | 3.86 | 1 | 2.74 | 3.8 |
| BnBs | Bs29 Tyr | Ala | 3.4 | 11.05 | 10.07 | 3.46 | −1 | 3.69 | 2 | 6.42 | 3.72 |
| BnBs | Bs35 Asp | Ala | 4.5 | 7.65 | 8.02 | 1.81 | 0 | 1.77 | 1 | 7.7 | 1.8 |
| BnBs | Bs38 Trp | Phe | 1.6 | −6.05 | −6.23 | 1.15 | 0 | −6.48 | 2 | −6.76 | −7.27 |
| BnBs | Bs39 Asp | Ala | 7.7 | 26.48 | 26.5 | −0.9 | −1 | 29.66 | 1 | 26.5 | 29.66 |
| BnBs | Bs42 Thr | Ala | 1.8 | 13.92 | 7.07 | −0.57 | −1 | 8.28 | 1 | 7.03 | 8.11 |
| BnBs | Bs44 Trp | Phe | 0 | 0.39 | 0.03 | 2.43 | 1 | −2.08 | 2 | 0.13 | −2.05 |
| BnBs | Bs76 Glu | Ala | 1.4 | 1.41 | 1.29 | 0.41 | 0 | 7.38 | 1 | 0.9 | 7.2 |
| BnBs | Bs80 Glu | Ala | 0.5 | −7.64 | −7.69 | −0.11 | 0 | −5.1 | 1 | −4.06 | −5.35 |
| HyHel-63 | L31 Asn | Ala | 2 | 8.45 | 8.45 | 1.3 | 0 | 6.6 | 2 | 5.1 | 2.78 |
| HyHel-63 | L32 Asn | Ala | 4.1 | 14.22 | 14.47 | 1.39 | 0 | 17.25 | 2 | 14.29 | 17.22 |
| HyHel-63 | L50 Tyr | Ala | 2.7 | 3.34 | 3.76 | 4.81 | 0 | −2.75 | 2 | 2.69 | −3.93 |
| HyHel-63 | L91 Ser | Ala | 1.4 | −1.45 | −2.06 | −0.86 | 0 | −2.39 | 1 | −2.1 | −2.32 |
| HyHel-63 | L96 Tyr | Ala | 1.1 | 1.88 | 1.93 | 0 | 0 | −0.42 | 2 | 2.02 | −0.36 |
| HyHel-63 | H32 Asp | Ala | 2 | 16.07 | 16.4 | −0.33 | −1 | 28.03 | 1 | 19.67 | 27.05 |
| HyHel-63 | H33 Tyr | Ala | 5.5 | 11.45 | 11.38 | 0.61 | −2 | 3.33 | 2 | 11.41 | 7.79 |
| HyHel-63 | H50 Tyr | Ala | 6.9 | 9.28 | 9.3 | 1.38 | 0 | 9.86 | 2 | 8.49 | 9.79 |
| HyHel-63 | H53 Tyr | Ala | 1.2 | 5.95 | 8.41 | −1.79 | −2 | 2.1 | 2 | 8.94 | −3.32 |
| HyHel-63 | H98 Trp | Ala | 4.9 | 5.72 | 5.79 | 6.3 | 1 | −2.93 | 2 | 6.41 | −2.27 |
| HyHel-63 | C20 Tyr | Ala | 3.3 | −3.04 | −1.74 | 6.48 | 0 | −8.13 | 2 | 0.94 | −3.21 |
| HyHel-63 | C21 Arg | Ala | 1.3 | 8.21 | 8.11 | 3.36 | 1 | −0.85 | 2 | 7.96 | −0.82 |
| HyHel-63 | C62 Trp | Ala | 0.8 | 4.67 | 0.85 | 1.58 | 0 | 0.19 | 2 | 0.76 | 0.15 |
| HyHel-63 | C63 trp | Ala | 1.3 | 5.29 | 5.34 | 1.07 | −3 | −0.45 | 2 | 3.51 | −3.23 |
| HyHel-63 | C75 Leu | Ala | 1.5 | 7.45 | 7.51 | 2.48 | −2 | 3.67 | 2 | 7.56 | 3.68 |
| HyHel-63 | C89 Thr | Ala | 0.8 | 1.69 | 1.8 | −1.97 | −1 | 0.94 | 1 | 1.68 | 0.08 |
| HyHel-63 | C93 Asn | Ala | 0.6 | 4.45 | 3.45 | 1.17 | 1 | 1.24 | 2 | 3.07 | 1.51 |
| HyHel-63 | C96 Lys | Ala | 6.1 | 12.8 | 13.39 | 3.66 | −1 | 13.42 | 2 | 13.6 | 13.45 |
| HyHel-63 | C97 Lys | Ala | 3.5 | 0.69 | 0.49 | 3.06 | −1 | 5.9 | 2 | 1.66 | 2.83 |
| HyHel-63 | C100 Ser | Ala | 0.8 | 1.05 | 1.22 | −0.33 | 0 | 0.84 | 1 | 1.7 | 1.28 |
| HyHel-63 | C101 Asp | Ala | 1.3 | 18.66 | 14.59 | −5.4 | −4 | 13.54 | 1 | 14.48 | 12.84 |

TABLE 9
Mutations in bH1-Herceptin Antibody-Antigen Complex

| | | | | | | EGAD | Egad | | | top | top |
|---|---|---|---|---|---|---|---|---|---|---|---|
| System | Position | Mutation | ΔΔGexp | ΔΔG* | ΔΔG** | stability | clashes | ΔΔEpol* | ΔCodon | ΔΔG | ΔΔEpol |
| anti-HER2 | L27 Gln | Ala | NA | 0.14 | 0.15 | −5.07 | −4 | 0.1 | 2 | 0.15 | 0.1 |
| anti-HER2 | L28 Asp | Ala | 0 | 0.02 | 0.03 | 0 | 0 | −0.08 | 1 | 0.1 | −0.01 |
| anti-HER2 | L29 Ile | Ala | −0.2 | tbd | tbd | 0.67 | 0 | tbd | 2 | tbd | tbd |
| anti-HER2 | L30 Pro | Ala | 0.1 | −0.18 | −0.16 | — | — | −0.07 | 1 | −0.17 | −0.07 |
| anti-HER2 | L30a Arg | Ala | −0.2 | 5.08 | 4.52 | −1.86 | −2 | 13.3 | 2 | 3.29 | 12.9 |
| anti-HER2 | L30b Ser | Ala | 0.3 | −0.49 | −0.42 | −0.05 | 0 | −0.26 | 1 | −0.16 | 0 |
| anti-HER2 | L30c Ile | Ala | 0.9 | −0.44 | −0.41 | 4.11 | 0 | −1.05 | 2 | −0.42 | −1.07 |
| anti-HER2 | L30d Ser | Ala | −0.2 | 0.07 | 0.22 | 0.02 | 0 | 0.09 | 1 | 0.31 | 0.19 |
| anti-HER2 | L31 Gly | Ala | 0.3 | −0.78 | −0.76 | — | — | −0.15 | 1 | −0.76 | −0.13 |
| anti-HER2 | L32 Tyr | Ala | −0.8 | 6.11 | 6.13 | −1.41 | −5 | −3.83 | 2 | 5.98 | −3.83 |
| anti-HER2 | L50 Trp | Ala | 1.4 | 4.38 | 4.56 | 5.93 | 0 | −2.35 | 2 | 4.53 | −2.35 |
| anti-HER2 | L51 Gly | Ala | −0.3 | −0.05 | −0.03 | — | — | −0.09 | 1 | −0.05 | −0.11 |
| anti-HER2 | L52 Ser | Ala | 0.3 | −9.15 | −9.07 | −0.4 | 0 | −17.08 | 1 | 0.11 | 0.12 |
| anti-HER2 | L53 Tyr | Ala | 0.9 | 2.33 | 2.33 | 2.09 | −1 | −1.76 | 2 | 2.25 | −1.76 |
| anti-HER2 | L91 His | Ala | 0.9 | 4.18 | 4.13 | 3.75 | 0 | 3.35 | 2 | 5.52 | 3.35 |
| anti-HER2 | L92 Tyr | Ala | 0.5 | 3.8 | 3.95 | 2.62 | −2 | −1.45 | 2 | 4.02 | −1.35 |
| anti-HER2 | L93 Thr | Ala | −0.4 | −0.49 | −0.47 | 1.69 | 0 | −0.77 | 1 | 1.1 | −0.8 |
| anti-HER2 | L94 Thr | Ala | 1.4 | 5.14 | 6.4 | −2.2 | 0 | 7.45 | 1 | 6.21 | 6.99 |
| anti-HER2 | H30 Lys | Ala | −0.3 | −0.32 | −0.33 | 0.82 | 0 | −0.4 | 2 | −0.25 | −0.32 |
| anti-HER2 | H31 Asp | Ala | 0.4 | 0.6 | 0.59 | 0.75 | 0 | 0.84 | 1 | 0.52 | 0.77 |
| anti-HER2 | H32 Thr | Ala | −0.4 | −0.06 | −0.04 | 1.86 | −1 | −0.13 | 1 | −0.04 | −0.13 |
| anti-HER2 | H33 Tyr | Ala | >2.4 | −2.57 | −2.75 | 4.25 | 0 | −6.07 | 2 | −3.44 | −4.21 |
| anti-HER2 | H50 Arg | Ala | >2.5 | 25.63 | 26.89 | 3.8 | 0 | 32.17 | 2 | 26.34 | 32.43 |
| anti-HER2 | H52 Tyr | Ala | −0.8 | −0.8 | −0.76 | −1.94 | 0 | −0.68 | 2 | −0.58 | −0.43 |
| anti-HER2 | H53 Thr | Ala | 0.1 | 0.13 | 0.16 | −3.13 | 0 | 0 | 1 | 0.3 | 0.15 |
| anti-HER2 | H54 Asn | Ala | −0.8 | −0.55 | −0.7 | −0.14 | −1 | −0.35 | 2 | 0.05 | 0.58 |
| anti-HER2 | H56 Tyr | Ala | >1.8 | 3.98 | 3.95 | 0.11 | −1 | 0.01 | 2 | 3.48 | −0.23 |
| anti-HER2 | H58 Arg | Ala | >2.5 | −995.06 | 2.39 | 1.32 | 0 | −1.76 | 2 | 0.93 | −1.32 |
| anti-HER2 | H95 Trp | Ala | >1.8 | 3.92 | 3.98 | 5.98 | 0 | 1.78 | 2 | 3.93 | 1.73 |
| anti-HER2 | H96 Gly | Ala | 0.1 | −0.15 | −0.14 | — | — | 0.03 | 1 | −0.13 | 0.03 |
| anti-HER2 | H97 Gly | Ala | 0.3 | −0.97 | −0.96 | — | — | −0.88 | 1 | −0.98 | −0.88 |
| anti-HER2 | H98 Asp | Ala | −0.1 | −3.16 | −2.78 | −0.34 | 0 | −1.93 | 1 | −0.32 | 1.2 |
| anti-HER2 | H99 Gly | Ala | 1.8 | −0.09 | −0.99 | − | — | −3.72 | 1 | −0.2 | −2.32 |
| anti-HER2 | H100 Phe | Ala | >1.9 | −0.55 | −0.56 | 4.09 | 0 | −0.33 | 2 | −0.3 | −0.28 |
| anti-HER2 | H100a Tyr | Ala | 2.2 | 6.29 | 6.42 | 2.43 | −1 | −0.11 | 2 | 6.41 | −0.11 |

1. A method of identifying a variant of an antibody with enhanced antigen binding affinity, the method comprising:
(a) determining a three-dimensional representation of an antibody-antigen interface of a first antibody,
(b) conformationally sampling point mutations of amino acid residues of the antibody at the antibody-antigen interface,
(c) selecting the point mutations of step (b) that are conformationally allowed,
(d) selecting the point mutations of step (c) that have both a Boltzmann averaged predictor based on a change of a change of a free energy of binding (ΔΔG*) of less than zero kcal/mol and a Boltzmann averaged predictor based on only a change of a change of the polar component of a free energy of binding (ΔΔEₚₒₗ*) of less than zero kcal/mol, and
(e) creating a focused library of antibodies containing at least one point mutation from the point mutations of step (d),
(f) screening the antibodies of step (e) for enhanced antigen-binding affinity in vitro; and
(g) selecting an antibody of step (f) having enhanced binding affinity relative to the first antibody, thereby identifying a variant of an antibody with enhanced antigen binding affinity.

2. The method of claim 1 wherein the amino acid residues of the antibody at the antibody-antigen interface that are conformationally sampled in step (b) are limited to those amino acid residues that are determined by in silico alanine screening to have an effect on the overall change in the change of free energy of binding of less than 1 kcal/mol.

3. The method of claim 1 wherein the amino acid residues of the antibody at the antibody-antigen interface that are conformationally sampled in step (b) are limited to those amino acid residues that are point mutations caused by at least two nucleic acid base changes in the codon coding for the point mutation of the antibody.

4. The method of claim 1 wherein the amino acid residues of the antibody at the antibody-antigen interface that are conformationally sampled in step (b) are limited to those amino acid residues that are point mutations that do not cause a change in the stability of the modified, mutated or altered antibody that is greater than 3 kcal/mol.

5. The method of claim 1, wherein the three-dimensional representation is a crystal structure having a resolution of about 2.5 Angstroms or less.

6. The method of claim 1, wherein the point mutations of the amino acid residues of step (b) result in an alteration of amino acid side chain chemistry.

7. The method of claim 6, further comprising expressing the modified, mutated or altered antibody.

8. The method of claim 1, wherein in the method is repeated at least one time.

9. The method of claim 1, wherein at least one step is informed by data selected from the group consisting of binding data derived from an expressed antibody binding to an antigen in an aqueous buffer, crystal structure data of an antibody, crystal structure data of an antibody bound to an antigen, three-dimensional structural data of an antibody, NMR structural data of an antibody, and computer-modeled structural data of an antibody.

10. The method of claim 7, wherein expressing the modified antibody is in an expression system selected from the group consisting of an acellular extract expression system, a phage display expression system, a prokaryotic cell expression system, and a eukaryotic cell expression system.

11. The method of claim 7, wherein the antibody, or antigen-binding fragment thereof, is modified at one or more CDR and/or framework positions within the light and/or heavy chain variable regions of the antibody or binding fragment.

12. The method of claim 7, wherein the antibody, or antigen-binding fragment thereof, is modified at one or more positions within a CDR region(s) selected from the group consisting of V_{H} CDR1, V_{H} CDR2, V_{H} CDR3, V_{L} CDR1, V_{L} CDR2, and V_{L} CDR3.

13. The method of claim 7, wherein the antibody, or antigen-binding fragment thereof, is selected from the group consisting of an antibody, an antibody light chain (V_{L}), an antibody heavy chain (V_{H}), a single chain antibody (scFv), a F(ab′)₂ fragment, a Fab fragment, an Fd fragment, and a single domain fragment.

14. The method of claim 1, wherein the antigen-binding affinity of the antibody is predicted to be increased by a factor of about 1.1, 1.2, 1.3, 1.4, 1.5, 1.6, 1.7, 1.8, 1.9, 2, 3, 5, 8, 10, 50, 10², 10³, 10⁴, 10⁵, 10⁶, 10⁷, or 10⁸.

15. A plurality of antibodies, or antigen-binding fragments thereof, produced by the method of claim 7.

16. A nucleic acid encoding the antibody, or antigen-binding fragment thereof, of claim 7.

17. A host cell comprising the nucleic acid of claim 16.

18. An antibody, or binding fragment thereof, produced by culturing the host cell of claim 17 under conditions such that antibody, or binding fragment thereof, is expressed.

19. A pharmaceutical composition comprising the antibody, or antigen-binding fragment thereof, of claim 18.

20. A method for treating or preventing a human disorder or disease comprising, administering a therapeutically-effective amount of the pharmaceutical composition of claim 19, such that therapy or prevention of the human disease or disorder is achieved.

21. A method of enhancing the antigen-binding affinity of an antibody comprising:
(a) determining a three-dimensional representation of an antibody-antigen interface,
(b) generating amino acid mutations of the antibody at the interface,
(c) generating a conformational search space of the mutations of step (b),
(d) comparing the conformational search space of (b) against a library of rotamers and organizing those mutations that have allowable conformational space as a search tree,
(e) pruning the search tree using the Dead End Elimination algorithm followed by the A* algorithm,
(f) calculating the lowest total energy of the remaining mutants of (e) by applying a molecular mechanics algorithm to the remaining mutants of (e),
(g) calculating the change in the change of the free energy of binding of the antibodies resulting from the mutants having conformations with the lowest total energy from (f) to an antigen by using a molecular mechanics Poisson-Boltzmann surface area algorithm on the antibodies of (f) bound to antigens,
(h) using an algorithm to evaluate the stability of the antibodies of step (g) having a change in the change of the free energy of binding of less than zero,
(i) restricting the number of antibodies generated from step (h) to antibodies having amino acid mutations caused by at least two changes in the nucleic acid bases of codons for the amino acid mutations of the antibodies of step (h),
(j) using the amino acid mutations of the antibodies of step (i) to design a focused library of point mutations for in vitro affinity maturation,
(k) selecting an amino acid residue of the antibody from the library of point mutations of step (j) for substitution in the antibody such that upon substitution, the antigen-binding affinity of the antibody is enhanced.

22. A method of identifying a variant of an enzyme with enhanced substrate binding affinity, the method comprising:
(a) determining a three-dimensional representation of an enzyme-substrate interface of a first enzyme,
(b) conformationally sampling point mutations of amino acid residues of the enzyme at the enzyme-substrate interface,
(c) selecting the point mutations of step (b) that are conformationally allowed,
(d) selecting the point mutations of step (c) that have both a Boltzmann averaged predictor based on a change of a change of a free energy of binding (ΔΔG*) of less than zero kcal/mol and a Boltzmann averaged predictor based on only a change of a change of the polar component of the free energy of binding (ΔΔEₚₒₗ*) of less than zero kcal/mol, and
(e) creating a library of enzymes containing at least one point mutation from the point mutations of step (d),
(f) screening the enzymes of step (e) for enhanced substrate-binding affinity in vitro; and
(g) selecting an enzyme of step (f) having enhanced substrate binding affinity relative to the first enzyme, thereby identifying a variant of an enzyme with enhanced substrate binding affinity.

23. The method of claim 22, wherein the amino acid residues of the enzyme at the enzyme-substrate interface that are conformationally sampled in step (b) are limited to those amino acid residues that are determined by in silico alanine screening to have an effect on the overall change in the change of free energy of binding of less than 1 kcal/mol.

24. The method of claim 22, wherein the amino acid residues of the enzyme at the enzyme-substrate interface that are conformationally sampled in step (b) are limited to those amino acid residues that are point mutations caused by at least two nucleic acid base changes in the codon coding for the point mutation of the enzyme.

25. The method of claim 22, wherein the amino acid residues of the enzyme at the enzyme-substrate interface that are conformationally sampled in step (b) are limited to those amino acid residues that are point mutations that do not cause a change in the stability of the modified, mutated or altered enzyme that is greater than 3 kcal/mol.

26. The method of claim 22, wherein the three-dimensional representation is a crystal structure having a resolution of about 2.5 Angstroms or less.

27. The method of claim 22, wherein the point mutations of the amino acid residues of step (b) comprise an alteration of the side chains of the amino acid residues.

28. The method of claim 27, further comprising expressing the modified, mutated or altered enzyme.

29. The method of claim 22, wherein in the method is repeated at least one time.

30. The method of claim 22, wherein at least one step is informed by data selected from the group consisting of substrate binding data derived from an expressed enzyme binding to a substrate in a solvent, crystal structure data of an enzyme, crystal structure data of an enzyme bound to a substrate, three-dimensional structural data of an enzyme, NMR structural data of an enzyme, and computer-modeled structural data of an enzyme.

31. The method of claim 28, wherein expressing the modified enzyme is in an expression system selected from the group consisting of an acellular extract expression system, a phage display expression system, a prokaryotic cell expression system, and a eukaryotic cell expression system.

32. The method of claim 28, wherein the enzyme is modified at one or more positions at the enzyme active site.

33. The method of claim 22, wherein the substrate binding affinity of the enzyme is predicted to be increased by a factor of about 1.1, 1.2, 1.3, 1.4, 1.5, 1.6, 1.7, 1.8, 1.9, 2, 3, 5, 8, 10, 50, 10², 10³, 10⁴, 10⁵, 10⁶, 10⁷, or 10⁸.

34. A plurality of enzymes produced by the method of claim 28.

35. A nucleic acid encoding the enzyme of claim 28.

36. A host cell encoding the nucleic acid of claim 35.

37. An enzyme produced by culturing the host cell of claim 36 under conditions such that the enzyme is expressed.

38. A pharmaceutical composition comprising the enzyme of claim 37.

39. A method for treating or preventing a human disorder or disease comprising, administering a therapeutically-effective amount of the pharmaceutical composition of claim 38, such that therapy or prevention of the human disease or disorder is achieved.

40. The method of claim 22, wherein the catalytic efficiency of the enzyme is increased through the point mutations of step (d).

41. The method of claim 1, wherein a generalized Born model is used instead of a Poisson Boltzmann model.

42. The method of claim 1, further comprising modeling the contribution of crystallographic waters to the free energy of binding of the antibodies of step (d) to an antigen.

43. The method of claim 1, further comprising modeling the antibody-antigen interface using a molecular dynamics algorithm to quantify the entropic part of the free energy of binding of the antibody to an antigen.

44. The method of claim 1, further comprising calculating the free energy of binding with the additional contribution to the free energy of binding of the antibody to an antigen from modeling the backbone flexibility of the antibody.
